(19) United States
(12) Patent Application Publication
(10) Pub. No.: US 2008/0064032 A1
(43) Pub. Date: Mar. 13, 2008

(54) Polynucleotides and uses thereof

(75) Inventors: Geoffrey Townshend, Cambridge (GB); Edward Hinchliffe, Macclesfield (GB); Andrew Dinsmore, Macclesfield (GB); Thomas Hohn, Research Trinangle Park, NC (US); Rene Quadt, Research Triangle Park, NC (US); Michele Susan Yarnall, Research Triangle Park, NC (US); Lilian Zeitouni, Raleigh, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 11/520,349

(22) Filed: Sep. 13, 2006

(51) Int. Cl. A01H 5/00; C07H 21/04; C12Q 1/68; G01N 33/53

(52) U.S. Cl. 435/6; 435/7.1; 536/23.6; 536/24.33; 800/320.3

(57) ABSTRACT

A novel transgenic wheat event designated JOPLIN1, is disclosed. The invention relates to DNA sequences of the recombinant constructs inserted into the wheat genome and of genomic sequences flanking the insertion site that resulted in the JOPLIN1 event. The invention further relates to assays for detecting the presence of the DNA sequences of JOPLIN1, to wheat plants and wheat seeds comprising the genotype of JOPLIN1 and to methods for producing a wheat plant by crossing a wheat plant comprising the JOPLIN1 genotype with itself or another wheat variety

This application claims the benefit of priority to U.S. Provisional Application Ser. No. 60/716,275, filed on Sep. 12, 2005.

### FIELD OF THE INVENTION

The present invention relates generally to the field of plant molecular biology, plant transformation, and plant breeding. More specifically, the invention relates to novel polynucleotides useful in identifying disease resistant transgenic wheat plants comprising a novel transgenic genotype and to methods of detecting the presence of the wheat plant DNA in a sample and compositions thereof.

### BACKGROUND

Numerous fungi are serious pests of economically important agricultural crops. Further, crop contamination by fungal toxins is a major problem for agriculture throughout the world. Mycotoxins are toxic fungal metabolites, often found in agricultural products, which are characterized by their ability to cause health problems for vertebrates. Trichothecenes are sesquiterpene epoxide mycotoxins produced by species of Fusarium, Trichothecium, and Myrothecium that act as potent inhibitors of eukaryotic protein synthesis. Fusarium species that produce such trichothecenes include F. acuminatum, F. crookwellense, F. culmorum, F. equiseti, F. graminearum (Gibberella zeae), F. lateritium, F. poae, F. sambucinum (G. pulicaris), and F. sporotrichioides (Marasas, W. F. O., Nelson, P. E., and Toussoun, T. A. 1984).

As previously described (A. E. Desjardins and T. M Hohn, Mycotoxins in plant pathogenesis. Mol.Plant-Microbe Interact. 10 (2):147-152, 1997), both acute and chronic mycotoxicoses in farm animals and in humans have been associated with consumption of wheat, rye, barley, oats, rice and maize contaminated with Fusarium species that produce trichothecene mycotoxins. Experiments with chemically pure trichothecenes at low dosage levels have reproduced many of the features observed in mouldy-grain toxicoses in animals, including anaemia and immunosuppression, haemorrhage, emesis and feed refusal. Historical and epidemiological data from human populations indicate an association between certain disease epidemics and consumption of grain infected with Fusarium species that produce trichothecenes. In particular, outbreaks of a fatal disease known as alimentary toxic aleukia, which has occurred in Russia since the nineteenth century, have been associated with consumption of over-wintered grains contaminated with Fusarium species that produce the trichothecene T-2 toxin. In Japan, outbreaks of a similar disease called akakabi-byo or red mould disease have been associated with grain infected with Fusarium species that produce the trichothecene, deoxynivalenol (hereinafter “DON”). Trichothecenes were detected in the toxic grain samples responsible for recent human disease outbreaks in India and Japan. There exists, therefore, a need for agricultural methods for preventing and, crops having reduced levels of, mycotoxin contamination.

Further, trichothecene-producing Fusarium species are destructive pathogens and attack a wide range of plant species. The acute phytotoxicity of trichothecenes and their occurrence in plant tissues also suggest that these mycotoxins play a role in the pathogenesis of Fusarium on plants. This implies that mycotoxins play a role in disease and, therefore, reducing their toxicity to the plant may also prevent or reduce disease in the plant. Further, reduction in disease levels may have the additional benefit of reducing mycotoxin contamination of the plant and particularly in grain where the plant is a cereal plant.

Various methods of controlling diseases in plants, such as wheat ear rot, stock rot or wheat head blight, have been used with varying degrees of success. One method of controlling plant disease has been to apply an antimicrobial chemical to crops. This method has numerous, art-recognized problems. Alternatively, a more recent method involves the use of biological control organisms (“biocontrol”) which are natural competitors or inhibitors of the pest organism. However, it is difficult to apply biocontrol to large areas, and even more difficult to cause those living organisms to remain in the treated area for an extended period of time. More recently, techniques in recombinant DNA have provided the opportunity to insert into plant cells, cloned genes that express antimicrobial compounds. However, this technology has given rise to concerns about eventual microbial resistance to well-known, naturally occurring antimicrobials. Thus, a continuing need exists to identify naturally occurring antimicrobial agents, such as proteins, which can be formed in plant cells directly by translation of a single gene.

A trichothecene 3-O-acetyltransferase that catalyses the acetylation of a number of different Fusarium trichothecenes including DON at the C3 hydroxyl group has been identified in F. sporotrichioides (S. P. McCormick, N. J. Alexander, S. C. Trapp, and T. M. Hohn. Disruption of TRI101, the gene encoding trichothecene 3-O-acetyltransferase, from Fusarium sporotrichioides. Applied.Environ.Microbiol. 65 (12):5252-5256, 1999). Acetylation of trichothecenes at the C3 hydroxyl group significantly reduces their toxicity in vertebrates and plants and results in the reaction product 3-acetyldeoxyvalenol (hereinafter “3ADON”), see Kimura et al. below.

The sequence of structural genes encoding trichothecene 3-O-acetyl transferases from Fusarium graminearum and Fusarium sporotrichioides, as well as sequences of other orthologs, has been published. See, e.g. Kimura et al., Biosci. Biotechnol. Biochem., 62(5): 1033-1036 (1998), and Kimura et al., FEBS Letters, 435:163-168 (1998). Further, it has been speculated that the gene from Fusarium sporotrichioides encoding a trichothecene 3-O-acetyl transferase may be useful in developing plant varieties with increased resistance to Fusarium. See e.g. Hohn, T. M. et al. Molecular Genetics of Host-Specific Toxins in Plant Disease, 17-24 (1998) and Kimura et al. J. Biological Chemistry, 273(3):1654-1661 (1998).

Wheat plants expressing these trichothecene 3-O-acetyltransferase genes have now been made and have been shown to be trichothecene and Fusarium resistant. The basic technology is described in U.S. Pat. Nos. 7,049,421, 6,646,184 and 6,346,655, all of which are herein incorporated by reference in their entirety.

The expression of foreign genes in plants can to be influenced by their chromosomal position, perhaps due to chromatin structure or the proximity of transcriptional regulation elements close to the integration site (See for example, Weising et al., 1988, “Foreign Genes in Plants,” Ann. Rev. Genet. 22:421-477). Therefore, it is common to produce hundreds of different events and screen those events for a single event that has desired transgene expression levels and patterns for commercial purposes. An event that has desired levels or patterns of transgene expression is useful for introgressing the transgene into other genetic backgrounds by sexual outcrossing using conventional breeding methods. Progeny of such crosses maintain the transgene expression characteristics of the original transformant. This strategy is used to ensure reliable gene expression in a number of varieties that are well adapted to local growing conditions.

It would be advantageous to be able to detect the presence of a particular event in order to determine whether progeny of a sexual cross contain a transgene of interest. In addition, a method for detecting a particular event would be helpful for complying with regulations requiring the pre-market approval and labeling of foods derived from recombinant crop plants, for example. It is possible to detect the presence of a transgene by any well-known nucleic acid detection method including but not limited to thermal amplification (polymerase chain reaction (PCR)) using polynucleotide primers or DNA hybridization using nucleic acid probes. Typically, for the sake of simplicity and uniformity of reagents and methodologies for use in detecting a particular DNA construct that has been used for transforming various plant varieties, these detection methods generally focus on frequently used genetic elements, for example, promoters, terminators, and marker genes, because for many DNA constructs, the coding sequence region is interchangeable. As a result, such methods may not be useful for discriminating between constructs that differ only with reference to the coding sequence. In addition, such methods may not be useful for discriminating between different events, particularly those produced using the same DNA construct unless the sequence of chromosomal DNA adjacent to the inserted heterologous DNA (“flanking DNA”) is known.

### SUMMARY

It is an object of the invention to provide polynucleotides that are useful in identifying a transgenic wheat event, designated JOPLIN1, comprising a novel transgenic genotype that comprises a trichothecene 3-O-acetyltransferase gene isolated from Fusarium sporotrichioides which confers disease resistance to the JOPLIN1 wheat event and progeny thereof. The invention also provides transgenic wheat plants comprising the genotype of the invention, seed from transgenic wheat plants comprising the genotype of the invention, and methods for producing a transgenic wheat plant comprising the genotype of the invention by crossing a wheat plant comprising the genotype of the invention with itself or another wheat plant of a different genotype. The present invention also provides compositions and methods for detecting the presence of polynucleotides from event JOPLIN1 based on the DNA sequence of the recombinant expression cassettes inserted into the wheat genome that resulted in the JOPLIN1 event and of genomic sequences flanking the insertion site. The present invention provides methods to further characterize the JOPLIN1 event by analyzing expression levels of trichothecene 3-O-acetyltransferase as well as by testing efficacy against wheat diseases.

According to one aspect, the present invention provides an isolated polynucleotide comprising at least 20 contiguous nucleotides from the wheat event JOPLIN1, wherein a first half of the contiguous nucleotides are heterologous DNA sequence inserted into the wheat plant genome of event JOPLIN1 and a second half of the contiguous nucleotides are wheat plant genome DNA flanking the point of insertion of a heterologous DNA sequence inserted into the wheat plant genome of event JOPLIN1. The isolated polynucleotide according to this aspect may comprise at least 30, 60, 120, 180 or at least 240 contiguous nucleotides from the wheat event JOPLIN1, wherein a first half of the contiguous nucleotides are heterologous DNA sequence inserted into the wheat plant genome of event JOPLIN1 and a second half of the contiguous nucleotides are wheat plant genome DNA flanking the point of insertion of a heterologous DNA sequence inserted into the wheat plant genome of event JOPLIN1. Still further provided is a polynucleotide which is the complement of the sequence described above.

In a further aspect, the present invention provides a polynucleotide which comprises at least 18 contiguous nucleotides of a nucleotide sequence selected from the group consisting of SEQ ID NO: 3, SEQ ID NO: 4, and complements thereof. Still further provided is a polynucleotide which comprises at least 20 contiguous nucleotides of a nucleotide sequence selected from the group consisting of SEQ ID NO: 3, SEQ ID NO: 4, and complements thereof. Still further provided is a polynucleotide which comprises at least 25 contiguous nucleotides of a nucleotide sequence selected from the group consisting of SEQ ID NO: 3, SEQ ID NO: 4, and complements thereof.

According to another aspect, the present invention provides an isolated nucleic acid molecule comprising a nucleotide sequence that comprises at least one junction sequence of event JOPLIN1 selected from the group consisting of SEQ ID NO: 3 and SEQ ID NO: 4, and complements thereof. A junction sequence spans the junction between the heterologous DNA comprising the expression cassettes inserted into the wheat genome and DNA from the wheat genome flanking the insertion site and is diagnostic for the JOPLIN1 event.

According to another aspect, the present invention provides a polynucleotide which comprises at least 35 contiguous nucleotides of the sequence of nucleotides 1364 to 1423 of SEQ ID NO: 1. Still further provided is a polynucleotide comprising at least 40 contiguous nucleotides of the sequence of nucleotides 1364 to 1423 of SEQ ID NO: 1. Still further provided is a polynucleotide comprising at least 50 contiguous nucleotides of the sequence of nucleotides 1364 to 1423 of SEQ ID NO: 1. Still further provided is a polynucleotide comprising nucleotides 1364 to 1423 of SEQ ID NO: 1. Still further provided is a polynucleotide which is the complement of the sequence described above.

According to another aspect, the present invention provides a polynucleotide comprising at least 70 contiguous nucleotides of the sequence of nucleotides 1334 to 1453 of SEQ ID NO: 1. Still further provided is a polynucleotide comprising at least 90 contiguous nucleotides of the sequence of nucleotides 1334 to 1453 of SEQ ID NO: 1. Still further provided is a polynucleotide comprising at least 100 contiguous nucleotides of the sequence of nucleotides 1334 to 1453 of SEQ ID NO: 1. Still further provided is a polynucleotide comprising nucleotides 1334 to 1453 of SEQ ID NO: 1. Still further provided is a polynucleotide which is the complement of the sequence described above.

According to another aspect, the present invention provides a polynucleotide comprising at least 110 contiguous nucleotides of the sequence of nucleotides 1304 to 1483 of SEQ ID NO: 1. Still further provided is a polynucleotide comprising at least 130 contiguous nucleotides of the sequence of nucleotides 1304 to 1483 of SEQ ID NO: 1. Still further provided is a polynucleotide comprising at least 150 contiguous nucleotides of the sequence of nucleotides 1304 to 1483 of SEQ ID NO: 1. Still further provided is a polynucleotide comprising nucleotides 1304 to 1483 of SEQ ID NO: 1. Still further provided is a polynucleotide which is the complement of the sequence described above.

According to another aspect, the present invention provides a polynucleotide comprising at least 160 contiguous nucleotides of the sequence of nucleotides 1274 to 1513 of SEQ ID NO: 1. Still further provided is a polynucleotide comprising at least 200 contiguous nucleotides of the sequence of nucleotides 1274 to 1513 of SEQ ID NO: 1. Still further provided is a polynucleotide comprising at least 220 contiguous nucleotides of the sequence of nucleotides 1274 to 1513 of SEQ ID NO: 1. Still further provided is a polynucleotide comprising nucleotides 1274 to 1513 of SEQ ID NO: 1. Still further provided is a polynucleotide which is the complement of the sequence described above.

According to another aspect, the present invention provides a polynucleotide which comprises at least 35 contiguous nucleotides of the sequence of nucleotides 397 to 456 of SEQ ID NO: 2. Still further provided is a polynucleotide which comprises at least 40 contiguous nucleotides of the sequence of nucleotides 397 to 456 of SEQ ID NO: 2. Still further provided is a polynucleotide which comprises at least 50 contiguous nucleotides of the sequence of nucleotides 397 to 456 of SEQ ID NO: 2. Still further provided is a polynucleotide which comprises nucleotides 397 to 456 of SEQ ID NO: 2. Still further provided is a polynucleotide which is the complement of the sequence described above.

According to another aspect, the present invention provides a polynucleotide which comprises at least 70 contiguous nucleotides of the sequence of nucleotides 367 to 486 of SEQ ID NO: 2. Still further provided is a polynucleotide which comprises at least 90 contiguous nucleotides of the sequence of nucleotides 367 to 486 of SEQ ID NO: 2. Still further provided is a polynucleotide which comprises at least 100 contiguous nucleotides of the sequence of nucleotides 367 to 486 of SEQ ID NO: 2. Still further provided is a polynucleotide which comprises nucleotides 367 to 486 of SEQ ID NO: 2. Still further provided is a polynucleotide which is the complement of the sequence described above.

According to another aspect, the present invention provides a polynucleotide which comprises at least 110 contiguous nucleotides of the sequence of nucleotides 337 to 516 of SEQ ID NO: 2. Still further provided is a polynucleotide which comprises at least 130 contiguous nucleotides of the sequence of nucleotides 337 to 516 of SEQ ID NO: 2. Still further provided is a polynucleotide which comprises at least 150 contiguous nucleotides of the sequence of nucleotides 337 to 516 of SEQ ID NO: 2. Still further provided is a polynucleotide which comprises nucleotides 337 to 5156 of SEQ ID NO: 2. Still further provided is a polynucleotide which is the complement of the sequence described above.

According to another aspect, the present invention provides an isolated nucleic acid molecule comprising a nucleotide sequence selected from the group consisting of SEQ ID NO: 1, SEQ ID NO: 2, and complements thereof.

According to another aspect of the invention, an amplicon comprising a polynucleotide of the invention is provided.

According to still another aspect of the invention, flanking sequence primers for detecting event JOPLIN1 are provided. Such flanking sequence primers comprise an isolated nucleic acid sequence comprising at least 10-15 contiguous nucleotides from a nucleotide sequence selected from the group consisting of nucleotides 1-1393 of SEQ ID NO: 1 (arbitrarily designated herein as the 5′ flanking sequence), nucleotides 427-2471 of SEQ ID NO: 2 (arbitrarily designated herein as the 3′ flanking sequence), or the complements thereof. In a further aspect the flanking sequence primer comprises the nucleotide sequence of SEQ ID NO: 6.

According to another aspect of the invention, primer pairs that are useful for nucleic acid amplification, for example, are provided. Such primer pairs comprise a first primer comprising a nucleotide sequence of at least 10-15 contiguous nucleotides in length which is or is complementary to one of the above-described genomic flanking sequences (SEQ ID NO: 1, or SEQ ID NO: 2) and a second primer comprising a nucleotide sequence of at least 10-15 contiguous nucleotides of heterologous DNA inserted into the event JOPLIN1 genome. The second primer may comprise a nucleotide sequence which is or is complementary to the insert sequence adjacent to the plant genomic flanking DNA sequence as set forth in SEQ ID NO: 1 from nucleotide position 1494 through 1788, in SEQ ID NO: 2 from nucleotide position 1 through 426 and in SEQ ID NO: 7 from nucleotide 4801 through 5512. In a further aspect, the second primer comprises the nucleotide sequence of SEQ ID NO: 5.

According to another aspect, the present invention provides a method for detecting a plant which comprises the JOPLIN1 genotype the method comprising: (a) preparing a sample containing the genomic DNA of the plant to be tested; (b) designing a pair of primers or a probe comprising a sufficient length of polynucleotides which is or is complementary to SEQ ID NO: 1, SEQ ID NO: 2, SEQ ID NO: 3, SEQ ID NO: 4, SEQ ID NO: 7, or SEQ ID NO: 8, or fragments of any of these sequences, wherein the primers or probe hybridize to isolated DNA from event JOPLIN1, and which, upon amplification of or hybridization to a nucleic acid sequence in the sample followed by detection of the amplicon or hybridization to the target sequence, are diagnostic for the presence of nucleic acid sequences from event JOPLIN1; (c) adding the pair of primers or probe to the sample and the means for performing an amplification reaction; (d) performing an amplification or hybridization reaction; and (e) visualizing the resulting amplicon or hybridized sequence, wherein the resulting amplicon or hybridized sequence identifies the plant as comprising the JOPLIN1 genotype.

According to another aspect of the invention, methods of detecting the presence of wheat event JOPLIN1 nucleic acids in a biological sample are provided. Such methods comprise: (a) contacting the sample comprising nucleic acids with a pair of primers that, when used in a nucleic acid amplification reaction with genomic DNA from wheat event JOPLIN1, produces an amplicon that is diagnostic for wheat event JOPLIN1; (b) performing a nucleic acid amplification reaction, thereby producing the amplicon; and (c) detecting the amplicon. In a further aspect, the amplicon comprises a nucleotide sequence selected from the group consisting of SEQ ID NO: 1, SEQ ID NO: 2, SEQ ID NO: 3, SEQ ID NO: 4, SEQ ID NO: 7, SEQ ID NO: 8, and the complements thereof.

According to another aspect, the invention provides methods of detecting the presence of wheat event JOPLIN1 nucleic acids in a biological sample. Such methods comprise: (a) contacting the sample comprising nucleic acids with a probe that hybridizes under high stringency conditions with genomic DNA from wheat event JOPLIN1 and does not hybridize under high stringency conditions with DNA of a control wheat plant; (b) subjecting the sample and probe to high stringency hybridization conditions; and (c) detecting hybridization of the probe to the nucleic acids. In a further aspect, the probe comprises a nucleotide sequence selected from the group consisting of SEQ ID NO: 1, SEQ ID NO: 2, SEQ ID NO: 3, SEQ ID NO: 4, SEQ ID NO: 7, SEQ ID NO: 8, and complements thereof.

According to another aspect of the invention, a kit is provided for the detection of wheat event JOPLIN1 nucleic acids in a biological sample. The kit includes at least one nucleic acid molecule comprising a sufficient length of polynucleotides which is or is complementary to SEQ ID NO: 1, SEQ ID NO: 2, SEQ ID NO: 3, SEQ ID NO: 4, SEQ ID NO: 7, or SEQ ID NO: 8, or fragments of any of these sequences, wherein the polynucleotides are useful as primers or probes that hybridize to isolated nucleic acids from wheat event JOPLIN1, and which, upon amplification of or hybridization to a nucleic acid sequence in a sample followed by detection of the amplicon or hybridization to the target sequence, are diagnostic for the presence of nucleic acid sequences from event JOPLIN1 in the sample. The kit further includes other materials necessary to enable nucleic acid hybridization or amplification methods.

In another aspect, the present invention provides a method of detecting wheat event JOPLIN1 protein in a biological sample comprising: (a) extracting protein from a sample of wheat event JOPLIN1 tissue; (b) assaying the extracted protein using an immunological method comprising antibody capable of binding to a trichothecene 3-O-acetyltransferase produced by the JOPLIN1 event; and (c) detecting the binding of the antibody to the trichothecene 3-O-acetyltransferase.

According to another aspect of the invention, wheat plants and seeds comprising the polynucleotides of the invention are provided.

The foregoing and other aspects of the invention will become more apparent from the following detailed description.

### DETAILED DESCRIPTION

The following definitions and methods are provided to better define the present invention and to guide those of ordinary skill in the art in the practice of the present invention. Unless otherwise noted, terms used herein are to be understood according to conventional usage by those of ordinary skill in the relevant art. Definitions of common terms in molecular biology may also be found in Rieger et al., Glossary of Genetics: Classical and Molecular, 5^{th} edition, Springer-Verlag: New York, 1994.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

“Detection kit” as used herein refers to a kit used to detect the presence or absence of DNA from JOPLIN1 plants in a sample comprising nucleic acid probes and primers of the present invention, which hybridize specifically under high stringency conditions to a target DNA sequence, and other materials necessary to enable nucleic acid hybridization or amplification methods.

As used herein the term transgenic “event” refers to a recombinant plant produced by transformation and regeneration of plant cells with heterologous DNA, i.e., a nucleic acid construct that includes a transgene of interest, regeneration of a population of plants resulting from the insertion of the transgene into the genome of the plant, and selection of a particular plant characterized by insertion into a particular location in the plant genome. The term “event” refers to the original transformant or progeny of the transformant, or both, that include the heterologous DNA. The term “event” also refers to progeny produced by a sexual outcross between the transformant and another variety that include the transgenic genotype. Even after repeated backcrossing to a recurrent parent, the inserted DNA and the flanking DNA from the transformed parent is present in the progeny of the cross at the same chromosomal location. The term “event” also refers to DNA from the original transformant and progeny thereof comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA. The transgenic wheat “event” of the present invention is designated JOPLIN1. Thus, “event JOPLIN1”, “JOPLIN1” or “JOPLIN1 event” as used herein, means the original JOPLIN1 transformant or progeny of the JOPLIN1 transformant, or both. Throughout this specification the term “Event 1” may be interchanged with the term “JOPLIN1.”

“Expression cassette” as used herein means a nucleic acid molecule capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette may also comprise sequences not necessary in the direct expression of a nucleotide sequence of interest but which are present due to convenient restriction sites for removal of the cassette from an expression vector. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation process known in the art. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development. An expression cassette, or fragment thereof, can also be referred to as “inserted sequence” or “insertion sequence” when transformed into a plant.

A “gene” is a defined region that is located within a genome and that, besides the aforementioned coding nucleic acid sequence, comprises other, primarily regulatory, nucleic acid sequences responsible for the control of the expression, that is to say the transcription and translation, of the coding portion. A gene may also comprise other 5′ and 3′ untranslated sequences and termination sequences. Further elements that may be present are, for example, introns.

“Gene of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

“Genotype” as used herein is the genetic material inherited from parent wheat plants not all of which is necessarily expressed in the descendant wheat plants. The JOPLIN1 genotype refers to the heterologous genetic material transformed into the genome of a plant as well as the genetic material flanking the inserted sequence.

A “heterologous” nucleic acid sequence is a nucleic acid sequence not naturally associated with a host cell into which it is introduced, including non-naturally occurring multiple copies of a naturally occurring nucleic acid sequence.

A “homologous” nucleic acid sequence is a nucleic acid sequence naturally associated with a host cell into which it is introduced.

“Operably-linked” refers to the association of nucleic acid sequences on a single nucleic acid fragment so that the function of one affects the function of the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences.

“Primers” as used herein are isolated nucleic acids that are annealed to a complimentary target DNA strand by nucleic acid hybridization to form a hybrid between the primer and the target DNA strand, then extended along the target DNA strand by a polymerase, such as DNA polymerase. Primer pairs or sets can be used for amplification of a nucleic acid molecule, for example, by the polymerase chain reaction (PCR) or other conventional nucleic-acid amplification methods.

A “probe” is an isolated nucleic acid to which is attached a conventional detectable label or reporter molecule, such as a radioactive isotope, ligand, chemiluminescent agent, or enzyme. Such a probe is complimentary to a strand of a target nucleic acid, in the case of the present invention, to a strand of genomic DNA from wheat event, JOPLIN1. The genomic DNA of JOPLIN1 can be from a wheat plant or from a sample that includes DNA from the event. Probes according to the present invention include not only deoxyribonucleic or ribonucleic acids but also polyamides and other probe materials that bind specifically to a target DNA sequence and can be used to detect the presence of that target DNA sequence.

Primers and probes are generally between 10 and 15 nucleotides or more in length, Primers and probes can also be at least 20 nucleotides or more in length, or at least 25 nucleotides or more, or at least 30 nucleotides or more in length. Such primers and probes hybridize specifically to a target sequence under high stringency hybridization conditions. Primers and probes according to the present invention may have complete sequence complementarity with the target sequence, although probes differing from the target sequence and which retain the ability to hybridize to target sequences may be designed by conventional methods.

“Stringent conditions” or “stringent hybridization conditions” include reference to conditions under which a probe will hybridize to its target sequence, to a detectably greater degree than to other sequences. Stringent conditions are target-sequence-dependent and will differ depending on the structure of the polynucleotide. By controlling the stringency of the hybridization and/or wash conditions, target sequences can be identified which are 100% complementary to the probe (homologous probing). Alternatively, stringency conditions can be adjusted to allow some mismatching in sequences so that lower degrees of similarity are detected (heterologous probing). Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, Part I, Chapter 2 “Overview of principles of hybridization and the strategy of nucleic acid probe assays”, Elsevier: N.Y.; and Current Protocols in Molecular Biology, Chapter 2, Ausubel et al., Eds., Greene Publishing and Wiley-Interscience: New York (1995), and also Sambrook et al. (2001) Molecular Cloning: A Laboratory Manual (5^{th} Ed. Cols Spring Harbor Laboratory, Cold Spring Harbor, N.Y.).

Specificity is typically the function of post-hybridization washes, the critical factors being the ionic strength and temperature of the final wash solution. Generally, high stringency hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, under high stringency conditions a probe will hybridize to its target subsequence, but to no other sequences.

An example of high stringency hybridization conditions for hybridization of complementary nucleic acids which have more than 100 complementary residues on a filter in a Southern or northern blot is 50% formamide with 1 mg of heparin at 42° C., with the hybridization being carried out overnight. An example of very high stringency wash conditions is 0.15M NaCl at 72° C. for about 15 minutes. An example of high stringency wash conditions is a 0.2×SSC wash at 65° C. for 15 minutes (see, Sambrook, infra, for a description of SSC buffer).

Exemplary hybridization conditions for the present invention include hybridization in 7% SDS, 0.25 M NaPO₄ pH 7.2 at 67° C. overnight, followed by two washings in 5% SDS, 0.20 M NaPO₄ pH7.2 at 65° C. for 30 minutes each wash, and two washings in 1% SDS, 0.20 M NaPO₄ pH7.2 at 65° C. for 30 minutes each wash. An exemplary medium stringency wash for a duplex of, e.g., more than 100 nucleotides, is 1×SSC at 45° C. for 15 minutes. An exemplary low stringency wash for a duplex of, e.g., more than 100 nucleotides, is 4-6×SSC at 40° C. for 15 minutes.

For probes of about 10 to 50 nucleotides, high stringency conditions typically involve salt concentrations of less than about 1.0 M Na ion, typically about 0.01 to 1.0 M Na ion concentration (or other salts) at pH 7.0 to 8.3, and the temperature is typically at least about 30° C. High stringency conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under high stringency conditions are still substantially identical if the proteins that they encode are substantially identical. This occurs, e.g., when a copy of a nucleic acid is created using the maximum codon degeneracy permitted by the genetic code.

The following are exemplary sets of hybridization/wash conditions that may be used to hybridize nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C. The sequences of the present invention may be detected using all the above conditions. For the purposes of defining the invention, the high stringency conditions are used.

“Transformation” is a process for introducing heterologous nucleic acid into a host cell or organism. In particular, “transformation” means the stable integration of a DNA molecule into the genome of an organism of interest.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule. As used herein, “transgenic” refers to a plant, plant cell, or multitude of structured or unstructured plant cells having integrated, via well known techniques of genetic manipulation and gene insertion, a sequence of nucleic acid representing a gene of interest into the plant genome, and typically into a chromosome of a cell nucleus, mitochondria or other organelle containing chromosomes, at a locus different to, or in a number of copies greater than, that normally present in the native plant or plant cell. Transgenic plants result from the manipulation and insertion of such nucleic acid sequences, as opposed to naturally occurring mutations, to produce a non-naturally occurring plant or a plant with a non-naturally occurring genotype. Techniques for transformation of plants and plant cells are well known in the art and may comprise for example electroporation, microinjection, Agrobacterium-mediated transformation, and ballistic transformation.

As used herein, the term “wheat” means Triticum aestivum (including spring, winter, and facultative wheat varieties) any other wheat species that can be bred with Triticum aestivum, including but not limited to durum wheat (Triticum durum), spelt (Triticum spelta), and emmer (Triticum dicoccum). Also encompassed are plants that are produced by conventional techniques using Triticum aestivum as a parent in a sexual cross with a non-Triticum species (such as rye [Secale cereale]), including but not limited to triticale.

The nomenclature for DNA bases and amino acids as set forth in 37 C.F.R. § 1.822 is used herein.

This invention relates to isolated polynucleotides useful in identifying a genetically improved line of wheat that produces the disease resistance protein, trichothecene 3-O-acetyltransferase. The invention is further drawn to a transgenic wheat event designated JOPLIN1 comprising a novel genotype, as well as to compositions and methods for detecting nucleic acids from this event in a biological sample. The invention is further drawn to wheat plants comprising the JOPLIN1 genotype, to transgenic seed from the wheat plants, and to methods for producing a wheat plant comprising the JOPLIN1 genotype by crossing a wheat plant comprising the JOPLIN1 genotype with itself or another wheat plant comprising a different genotype. Wheat plants comprising the JOPLIN1 genotype of the invention are useful in controlling diseases including Fusarium. The transgenic wheat plants of the invention may have essentially all of the morphological and physiological characteristics of the corresponding isogenic non-transgenic wheat plant in addition to those conferred upon the wheat plant by the novel genotype of the invention.

In one embodiment, the present invention encompasses an isolated polynucleotide comprising at least 20 or more (for example 30, 60, 120, 180, 240 or more) contiguous nucleotides from the wheat event JOPLIN1, wherein a first half of the contiguous nucleotides are heterologous DNA sequence inserted into the wheat plant genome of event JOPLIN1 and a second half of the contiguous nucleotides are wheat plant genome DNA flanking the point of insertion of a heterologous DNA sequence inserted into the wheat plant genome of event JOPLIN1. In a further embodiment the isolated polynucleotide may comprise at least 30, 60, 120, 180 or at least 240 contiguous nucleotides from the wheat event JOPLIN1, wherein a first half of the contiguous nucleotides are heterologous DNA sequence inserted into the wheat plant genome of event JOPLIN1 and a second half of the contiguous nucleotides are wheat plant genome DNA flanking the point of insertion of a heterologous DNA sequence inserted into the wheat plant genome of event JOPLIN1. Still further provided is a polynucleotide which is the complement of the sequence described above. Also included are nucleotide sequences that comprise 10 or more nucleotides of contiguous insert sequence from event JOPLIN1 and at least one nucleotide of flanking DNA from event JOPLIN1 adjacent to the insert sequence. Such nucleotide sequences are diagnostic for event JOPLIN1. Nucleic acid amplification of genomic DNA from the JOPLIN1 event produces an amplicon comprising such diagnostic nucleotide sequences.

In another embodiment, the invention encompasses an isolated nucleic acid molecule comprising a nucleotide sequence which comprises at least one junction sequence of event JOPLIN1 selected from the group consisting of SEQ ID NO: 3, SEQ ID NO: 4, and complements thereof, wherein a junction sequence spans the junction between a heterologous expression cassette inserted into the wheat genome and DNA from the wheat genome flanking the insertion site and is diagnostic for the JOPLIN1 event. In another embodiment, the junction sequence comprises as little as two nucleotides: those being the first nucleotide within the flanking genomic DNA adjacent to and covalently linked to the first nucleotide within the inserted heterologous DNA sequence. In a further embodiment the 5′ junction sequence comprises nucleotides 1393 and 1394 (c-c) of SEQ ID NO: 1 and the 3′ junction sequence comprises nucleotides 426 and 427 (c-g) of SEQ ID NO: 2.

In another embodiment, the invention encompasses an isolated polynucleotide comprising a nucleotide sequence selected from the group consisting of SEQ ID NO: 1, SEQ ID NO: 2, SEQ ID NO: 3, SEQ ID NO: 4, SEQ ID NO: 7, SEQ ID NO: 8, and the complements thereof.

In one embodiment of the present invention, an amplicon comprising a nucleotide sequence selected from the group consisting of SEQ ID NO: 1, SEQ ID NO: 2, SEQ ID NO: 3, SEQ ID NO: 4, SEQ ID NO: 7, SEQ ID NO: 8, and the complements thereof is provided.

In another embodiment, the present invention encompasses flanking sequence primers for detecting event JOPLIN1 nucleic acids. Such flanking sequence primers comprise an isolated nucleic acid sequence comprising at least 10-15 contiguous nucleotides from nucleotides 1-1393 of SEQ ID NO: 1 (arbitrarily designated herein as the 5′ flanking sequence), or the complements thereof.

In another embodiment, the present invention encompasses flanking sequence primers that comprise at least 10-15 contiguous nucleotides from nucleotides 427-2471 of SEQ ID NO: 2 (arbitrarily designated herein as the 3′ flanking sequence), or the complements thereof. In one aspect of this embodiment the flanking sequence primers comprise the nucleotide sequence of SEQ ID NO: 6 or the complement thereof.

In still another embodiment, the present invention encompasses a pair of polynucleotide primers comprising a first polynucleotide primer and a second polynucleotide primer which function together in the presence of a wheat event JOPLIN1 DNA template in a sample to produce an amplicon diagnostic for the wheat event JOPLIN1, wherein the first primer sequence is or is complementary to a wheat plant genome flanking the point of insertion of a heterologous DNA sequence inserted into the wheat plant genome of wheat event JOPLIN1, and the second polynucleotide primer sequence is or is complementary to the heterologous DNA sequence inserted into the wheat plant genome of the wheat event JOPLIN1.

In one aspect of this embodiment the first polynucleotide primer comprises at least 10 contiguous nucleotides from position 1-1393 of SEQ ID NO: 1, or complements thereof, or from position 427-2471 of SEQ ID NO: 2, or complements thereof. In another aspect of this embodiment, the first polynucleotide primer comprises the nucleotide sequence of SEQ ID NO: 6, or the complement thereof.

In yet another embodiment, the second polynucleotide primer comprises at least 10 contiguous nucleotides from positions 1394-1788 of SEQ ID NO: 1, or the complements thereof. In another embodiment, the second polynucleotide primer comprises the nucleotide sequence of SEQ ID NO: 5 or the complement thereof.

In another embodiment, the first polynucleotide primer, which is set forth in SEQ ID NO: 6, and the second polynucleotide primer which is set forth in SEQ ID NO: 5, function together in the presence of a wheat event JOPLIN1 DNA template in a sample to produce an amplicon diagnostic for the wheat event JOPLIN1.

It is well within the skill in the art to obtain additional sequence further out into the genome sequence flanking either end of the inserted heterologous DNA sequences for use as a primer sequence that can be used in such primer pairs for amplifying the sequences that are diagnostic for the JOPLIN1 event. For the purposes of this disclosure, the phrase “further out into the genome sequence flanking either end of the inserted heterologous DNA sequences” refers specifically to a sequential movement away from the ends of the inserted heterologous DNA sequences, the points at which the inserted DNA sequences are adjacent to native genomic DNA sequence, and out into the genomic DNA of the particular chromosome into which the heterologous DNA sequences were inserted. Preferably, a primer sequence corresponding to or complementary to a part of the insert sequence should prime the transcriptional extension of a nascent strand of DNA or RNA toward the nearest flanking sequence junction. Consequently, a primer sequence corresponding to or complementary to a part of the genomic flanking sequence should prime the transcriptional extension of a nascent strand of DNA or RNA toward the nearest flanking sequence junction. A primer sequence can be, or can be complementary to, a heterologous DNA sequence inserted into the chromosome of the plant, or a genomic flanking sequence. One skilled in the art would readily recognize the benefit of whether a primer sequence would need to be, or would need to be complementary to, the sequence as set forth within the inserted heterologous DNA sequence or as set forth in SEQ ID NO: 1 or SEQ ID NO: 2 depending upon the nature of the product desired to be obtained through the use of the nested set of primers intended for use in amplifying a particular flanking sequence containing the junction between the genomic DNA sequence and the inserted heterologous DNA sequence.

In one embodiment, the present invention encompasses a method of detecting the presence of wheat event JOPLIN1 nucleic acids in a biological sample, wherein the method comprises: (a) contacting the sample comprising nucleic acids with a pair of primers that, when used in a nucleic acid amplification reaction with genomic DNA from wheat event JOPLIN1 produces an amplicon that is diagnostic for wheat event JOPLIN1; (b) performing a nucleic acid amplification reaction, thereby producing an amplicon; and (c) detecting the amplicon. In a further embodiment, the amplicon comprises a nucleotide sequence selected from the group consisting of SEQ ID NO: 1, SEQ ID NO: 2, SEQ ID NO: 3, SEQ ID NO: 4, SEQ ID NO: 7, SEQ ID NO: 8, and the complements thereof.

In another embodiment, the present invention encompasses a method of detecting the presence of wheat event JOPLIN1 nucleic acids in a biological sample, wherein the method comprises: (a) contacting the sample comprising nucleic acids with a probe that hybridizes under high stringency conditions with genomic DNA from wheat event JOPLIN1 and does not hybridize under high stringency conditions with DNA of a control wheat plant; (b) subjecting the sample and probe to high stringency hybridization conditions; and (c) detecting hybridization of the probe to the nucleic acid. In a further embodiment the amplicon comprises a nucleotide sequence selected from the group consisting of SEQ ID NO: 1, SEQ ID NO: 2, SEQ ID NO: 3, SEQ ID NO: 4, SEQ ID NO: 7, SEQ ID NO: 8, and the complements thereof.

Detection can be by any means well known in the art including but not limited to fluorescent, chemiluminescent, radiological, immunological, or otherwise. In the case in which hybridization is intended to be used as a means for amplification of a particular sequence to produce an amplicon which is diagnostic for the JOPLIN1 wheat event, the production and detection by any means well known in the art of the amplicon is intended to be indicative of the intended hybridization to the target sequence where one probe or primer is utilized, or sequences where two or more probes or primers are utilized. The term “biological sample” is intended to comprise a sample that contains or is suspected of containing a nucleic acid comprising from between five and ten nucleotides either side of the point at which one or the other of the two terminal ends of the inserted heterologous DNA sequence contacts the genomic DNA sequence within the chromosome into which the heterologous DNA sequence was inserted, herein also known as the junction sequences.

In another embodiment, the present invention encompasses a method for detecting a plant which comprises the JOPLIN1 genotype the method comprising: (a) preparing a sample containing genomic DNA of the plant to be tested; (b) designing a pair of primers or a probe comprising a sufficient length of polynucleotides which is or is complementary to SEQ ID NO: 1, SEQ ID NO: 2, SEQ ID NO: 3, SEQ ID NO: 4, SEQ ID NO: 7, or SEQ ID NO: 8, or fragments of any of these sequences, wherein the primers or probe hybridize to isolated nucleic acids from event JOPLIN1, and which, upon amplification of or hybridization to a nucleic acid sequence in the sample followed by detection of the amplicon or hybridization to the target sequence, are diagnostic for the presence of nucleic acid sequences from event JOPLIN1; (c) adding the pair of primers or probe to the sample and the means for performing the amplification or hybridization reaction; (d) performing an amplification or hybridization reaction; and (e) visualizing the resulting amplicon or hybridized sequence, wherein the resulting amplicon or hybridized sequence identifies the plant as comprising the JOPLIN1 genotype.

In another embodiment, the present invention encompasses a method of detecting a plant comprising SEQ ID NO: 1 the method comprising: (a) preparing a sample containing genomic DNA of the plant to be tested; (b) designing a pair of primers which are suitable for use in an amplification reaction to amplify a sequence comprising at least 18 contiguous nucleotides of SEQ ID NO: 3 and the complement thereof; (c) adding the pair of primers to the sample and the means for performing an amplification reaction; (d) performing an amplification reaction; and (e) visualizing the resulting amplified sequence. In another embodiment the pair of primers are capable of amplifying a sequence comprising at least 20 or 25 contiguous nucleotides of SEQ ID NO: 3 and the complement thereof. In a further aspect, the primers are capable of amplifying a sequence comprising SEQ ID NO: 3.

In yet another embodiment, the present invention encompasses a method as described above wherein the sequence to be amplified by the amplification reaction comprises the junction of genomic sequence-insert provided as nucleotides 1393 and 1394 of SEQ ID NO: 1. A person skilled in the art will appreciate that this junction can be used to characterize and thus identify the event and so it is well within the ambit of the skilled person to design and produce oligonucleotide primer sequences that are suitable for use in an amplification reaction to amplify the sequence which comprises the junction. For example, the amplification product may comprise a small region of the genomic sequence, which genomic sequence is indicated as comprising nucleotides 1 to 1393 of SEQ ID NO: 1 and a larger region of the insert sequence, which insert sequence is indicated as the sequence comprising nucleotides 1394 to 1788 of SEQ ID NO: 1. Alternatively, the amplification product may comprise a small region of the insert sequence and a larger region of the genomic sequence. The person skilled in the art will also appreciate that the primer sequences suitable for use in an amplification reaction may be designed based on the genomic sequence which is 5′ i.e. upstream of nucleotide number 1 of SEQ ID NO: 1 and the insert or genomic sequence which is 3′ i.e. downstream of nucleotide number 1788 of SEQ ID NO: 1.

In another embodiment, the present invention provides a method for detecting a plant which comprises SEQ ID NO: 1 the method comprising: (a) preparing a sample containing the genomic DNA of the plant to be tested; and (b) designing a pair of primers which are suitable for use in an amplification reaction to amplify a sequence comprising at least 35 contiguous nucleotides of the sequence depicted as nucleotides 1364 to 1423 of SEQ ID NO: 1 and the complement thereof; and (c) adding the pair of primers to the sample and the means for performing an amplification reaction; and (d) performing an amplification reaction; and (e) visualizing the thus amplified sequence.

In another embodiment the primers are suitable for use in an amplification reaction to amplify a sequence comprising at least 40 contiguous nucleotides of the sequence of nucleotides 1364 to 1423 of SEQ ID NO: 1. In a further embodiment the primers are suitable for use in an amplification reaction to amplify a sequence comprising at least 50 contiguous nucleotides of the sequence of nucleotides 1364 to 1423 of SEQ ID NO: 1. In a further embodiment the primers are suitable for use in an amplification reaction to amplify a sequence comprising the sequence of nucleotides 1364 to 1423 of SEQ ID NO: 1.

In another embodiment the primers are suitable for use in an amplification reaction to amplify a sequence comprising at least 70 contiguous nucleotides of the sequence depicted as nucleotides 1334 to 1453 of SEQ ID NO: 1. In a further embodiment the primers are suitable for use in an amplification reaction to amplify a sequence comprising at least 90 contiguous nucleotides of the sequence of nucleotides 1334 to 1453 of SEQ ID NO: 1. In a further aspect the primers are suitable for use in an amplification reaction to amplify a sequence comprising at least 100 contiguous nucleotides of the sequence of nucleotides 1334 to 1453 of SEQ ID NO: 1. In a further embodiment the primers are suitable for use in an amplification reaction to amplify a sequence comprising nucleotides 1334 to 1453 of SEQ ID NO: 1.

In another embodiment the primers are suitable for use in an amplification reaction to amplify a sequence comprising at least 110 contiguous nucleotides of the sequence of nucleotides 1304 to 1483 of SEQ ID NO: 1. In a further embodiment the primers are suitable for use in an amplification reaction to amplify a sequence comprising at least 130 contiguous nucleotides of the sequence of nucleotides 1304 to 1483 of SEQ ID NO: 1. In a further embodiment the primers are suitable for use in an amplification reaction to amplify a sequence comprising at least 150 contiguous nucleotides of the sequence of nucleotides 1304 to 1483 of SEQ ID NO: 1. In a further embodiment the primers are suitable for use in an amplification reaction to amplify a sequence comprising nucleotides 1304 to 1483 of SEQ ID NO: 1.

In a further embodiment the primers are suitable for use in an amplification reaction to amplify a sequence comprising at least 160 contiguous nucleotides of the sequence of nucleotides 1274 to 1513 of SEQ ID NO: 1. In a further embodiment the primers are suitable for use in an amplification reaction to amplify a sequence comprising at least 200 contiguous nucleotides of the sequence of nucleotides 1274 to 1513 of SEQ ID NO: 1. In a further embodiment the primers are suitable for use in an amplification reaction to amplify a sequence comprising at least 220 contiguous nucleotides of the sequence of nucleotides 1274 to 1513 of SEQ ID NO: 1. In a further embodiment the primers are suitable for use in an amplification reaction to amplify a sequence comprising nucleotides 1274 to 1513 of SEQ ID NO: 1.

In another embodiment, the present invention encompasses a method for detecting a plant which comprises SEQ ID NO: 2 the method comprising: (a) preparing a sample containing the genomic DNA of the plant to be tested; (b) designing a pair of primers which are suitable for use in an amplification reaction to amplify a sequence comprising at least 18 contiguous nucleotides of the sequence depicted as SEQ ID NO: 4 and the complement thereof; (c) adding the pair of primers to the sample and the means for performing an amplification reaction; (d) performing an amplification reaction; and (e) visualizing the thus amplified sequence.

In one embodiment the pair of primers are capable of amplifying a sequence comprising at least 20 or 25 contiguous nucleotides of SEQ ID NO: 4 and the complement thereof. In a further embodiment, the primers are capable of amplifying a sequence comprising SEQ ID NO: 4.

The present invention still further encompasses a method as described above wherein the sequence to be amplified by the amplification reaction comprises the junction of genomic sequence-insert provided as nucleotides 426 and 427 of SEQ ID NO: 2. The person skilled in the art will appreciate that this junction can be used to characterize and thus identify the event and so it is well within the ambit of the skilled person to design and produce oligonucleotide primer sequences that are suitable for use in an amplification reaction to amplify the sequence which comprises the junction. For example, the amplification product may comprise a small region of the genomic sequence, which genomic sequence is indicated as comprising nucleotides 427 to 2471 of SEQ ID NO: 2 and a larger region of the insert sequence, which insert sequence is indicated as the sequence comprising nucleotides 1 to 426 of SEQ ID NO: 2. Alternatively, the amplification product may comprise a small region of the insert sequence and a larger region of the genomic sequence. The person skilled in the art will also appreciate that the primer sequences suitable for use in an amplification reaction may be designed based on the insert or genomic sequence which is 5′ i.e. upstream of nucleotide number 1 of SEQ ID NO: 2 and the genomic sequence which is 3′ i.e. downstream of the genomic sequence nucleotide number 427 of SEQ ID NO: 2.

In another embodiment, the present invention encompasses a method for detecting a plant which comprises SEQ ID NO: 2 the method comprising: (a) preparing a sample containing the genomic DNA of the plant to be tested; and (b) designing a pair of primers which are suitable for use in an amplification reaction to amplify a sequence comprising at least 35 contiguous nucleotides of the sequence depicted as nucleotides 397 to 456 of SEQ ID NO: 2 and the complement thereof; and (c) adding the pair of primers to the sample and the means for performing an amplification reaction; and (d) performing an amplification reaction; and (e) visualizing the thus amplified sequence. In one embodiment the primers are suitable for use in an amplification reaction to amplify a sequence comprising at least 40 contiguous nucleotides of the sequence depicted as nucleotides 397 to 456 of SEQ ID NO: 2. In a further embodiment the primers are suitable for use in an amplification reaction to amplify a sequence comprising at least 50 contiguous nucleotides of the sequence depicted as nucleotides 397 to 456 of SEQ ID NO: 2. In a further embodiment the primers are suitable for use in an amplification reaction to amplify a sequence comprising nucleotides 397 to 456 of SEQ ID NO: 2.

In a further embodiment the primers are suitable for use in an amplification reaction to amplify a sequence comprising at least 70 contiguous nucleotides of the sequence depicted as nucleotides 367 to 486 of SEQ ID NO: 2. In one embodiment the primers are suitable for use in an amplification reaction to amplify a sequence comprising at least 90 contiguous nucleotides of the sequence depicted as nucleotides 367 to 486 of SEQ ID NO: 2. In a further embodiment the primers are suitable for use in an amplification reaction to amplify a sequence comprising at least 100 contiguous nucleotides of the sequence depicted as nucleotides 367 to 486 of SEQ ID NO: 2. In a further embodiment the primers are suitable for use in an amplification reaction to amplify a sequence comprising nucleotides 367 to 486 of SEQ ID NO: 2.

In a further embodiment the primers are suitable for use in an amplification reaction to amplify a sequence comprising at least 110 contiguous nucleotides of the sequence depicted as nucleotides 337 to 516 of SEQ ID NO: 2. In one embodiment the primers are suitable for use in an amplification reaction to amplify a sequence comprising at least 130 contiguous nucleotides of the sequence depicted as nucleotides 337 to 516 of SEQ ID NO: 2. In a further embodiment the primers are suitable for use in an amplification reaction to amplify a sequence comprising at least 150 contiguous nucleotides of the sequence depicted as nucleotides 337 to 516 of SEQ ID NO: 2. In a further embodiment the primers are suitable for use in an amplification reaction to amplify a sequence comprising nucleotides 337 to 516 of SEQ ID NO: 2.

In yet another embodiment, the present invention encompasses a kit for detecting the presence of JOPLIN1 nucleic acids in a biological sample, wherein the kit comprises at least one nucleic acid molecule of sufficient length of contiguous nucleotides homologous or complementary to a nucleotide sequence selected from the group consisting of SEQ ID NO: 1, SEQ ID NO: 2, SEQ ID NO: 3, and SEQ ID NO: 4, that functions as a DNA primer or probe specific for event JOPLIN1, and other materials necessary to enable nucleic acid hybridization or amplification. A variety of detection methods can be used including TAQMAN (Perkin Elmer), thermal amplification, ligase chain reaction, southern hybridization, ELISA methods, and colorimetric and fluorescent detection methods. In particular the present invention provides for kits for detecting the presence of the target sequence, i.e., at least one of the junctions of the insert DNA with the genomic DNA of the wheat plant in JOPLIN1, in a sample containing genomic nucleic acid from JOPLIN1. The kit is comprised of at least one polynucleotide capable of binding to the target site or substantially adjacent to the target site and at least one means for detecting the binding of the polynucleotide to the target site. The detecting means can be fluorescent, chemiluminescent, colorimetric, or isotopic and can be coupled at least with immunological methods for detecting the binding. A kit is also envisioned which can detect the presence of the target site in a sample, i.e., at least one of the junctions of the insert DNA with the genomic DNA of the wheat plant in JOPLIN1, taking advantage of two or more polynucleotide sequences which together are capable of binding to nucleotide sequences adjacent to or within about 100 base pairs, or within about 200 base pairs, or within about 500 base pairs or within about 1000 base pairs of the target sequence and which can be extended toward each other to form an amplicon which contains at least the target site

In another embodiment, the present invention encompasses a method for detecting event JOPLIN1 trichothecene 3-O-acetyltransferase in a biological sample, the method comprising: (a) extracting protein from a sample of wheat event JOPLIN1 tissue; (b) assaying the extracted protein using an immunological method comprising an antibody capable of detecting a trichothecene 3-O-acetyltransferase produced by the JOPLIN1 event; and (c) detecting the binding of said antibody to the trichothecene 3-O-acetyltransferase.

Suitable methods of detecting plant material derived from the JOPLIN1 event described herein which methods are based on the antibody binding include, but are not limited to Western Blots, Enzyme-Linked ImmunoSorbent Assays (ELISA) and SELDI mass spectrometry. The skilled person is familiar with these and further immunological techniques. Typical steps include incubating a sample with an antibody that binds to the protein, washing to remove unbound antibody, and detecting whether the antibody has bound. Many such detection methods are based on enzymatic reactions—for example the antibody may be tagged with an enzyme such as horseradish peroxidase, and on application of a suitable substrate, a color change detected. Suitable antibodies may be monoclonal or polyclonal.

In another embodiment, the present invention encompasses a method of detecting JOPLIN1 plant material described herein the method comprising obtaining a sample for analysis; making a protein extract of the sample; providing a test strip or dipstick designed to detect the presence of a the protein present within the sample; incubating the test strip or dipstick with the sample; and detecting whether the protein is present.

In yet another embodiment of the method described above, typical steps include incubating a test strip or dipstick with a sample and observing the presence or absence of colored bands on the test strip or dipstick. The colored bands are indicative of the presence of a protein in the sample. Such test strip or dipstick tests are usually protein specific, and may be used for rapid testing of samples in the field.

In one embodiment, the dipstick utilizes an antibody or antibodies, or fragment/fragments thereof, specific for the trichothecene 3-O-acetyltransferase from F. sporotrichioides as encoded by nucleotides 3591 to 4970 of SEQ ID NO: 7. Antibody fragments include, but are not limited to, Fab, modified Fab, diFab, Fab′, F(ab′)2 or FV fragment, immunoglobulin light chain or heavy chain monomer, single chain FV (scFV) or nanobody. The antibody or fragment thereof may be monoclonal or polyclonal. In a particular embodiment, the antibody is an antibody secreted by cell lines selected from the group consisting of TR1-R Mab70 (Accession No. DSM ACC 2679) and TR1-R Mab72 (Accession No. DSM ACC 2680) or an antibody which is capable of inhibiting the binding to the trichothecene 3-O-acetyltransferase of an antibody secreted by cell lines selected from the group consisting of DSM ACC 2679 and DSM ACC 2680. It is noted that methods for producing both monoclonal and polyclonal antibodies and fragments thereof are well known in the art.

Suitable test strips or dipsticks and materials for their use are described in PCT application WO 02/27322 and are, for example, lateral-flow immunostrips comprising a detection membrane of cellulose acetate, cellulose, nitrocellulose or nylon, supported on a plastic backing. Such an immunostrip may be produced using membranes and filters through which a liquid sample is drawn by capillary action. The protein in the sample reacts with the antibodies contained in the immunostrip as it moves the length of the strip and is captured at a line that becomes visible. Suitable means of detection are, for example, colloidal gold and coloured latex beads.

In a further embodiment, a line of specific anti-trichothecene 3-O-acetyltransferase antibody, as described above, is sprayed on a test strip, which is suitably made from nitrocelluose supported on a plastic backing. A reagent control line of anti-mouse antibody is sprayed in parallel above the first antibody line. The membrane is flanked on the top by an absorption pad and on the bottom by a pad containing dried colloidal gold labeled anti-trichothecene 3-O-acetyltransferase antibody. In a preferred embodiment, the colloidal gold-labeled anti-trichothecene 3-O-acetyl transferase antibody is different from the first antibody sprayed as the test line. In a further embodiment, the colloidal gold-labeled anti-trichothecene 3-O-acetyl transferase antibody is the antibody secreted by cell line DSM ACC 2679 and the antibody sprayed at the test line is the antibody secreted by cell line DSM ACC 2680. A sample application pad flanks the colloidal gold pad. In use, the sample application pad is placed in a sample of extracted tissue or this sample is applied to the pad in another way, for example, by pipette. Any trichothecene 3-O-acetyltransferase protein contained within the sample flows up the strip and becomes bound by the colloidal gold labeled-anti-trichothecene 3-O-acetyltransferase antibody. As it continues up the strip, the protein also becomes bound by the anti-trichothecene 3-O-acetyltransferase antibody at the test line. Excess gold conjugate is captured at the reagent control line. In a positive test, that is, if trichothecene 3-O-acetyltransferase is present in the sample, a double red line appears: the lower line indicates the presence of trichothecene 3-O-acetyltransferase while the upper line is the control line signaling a properly working device.

In one embodiment, the present invention provides a wheat plant, wherein the JOPLIN1 genotype confers upon the wheat plant resistance to diseases. In a further embodiment, the genotype conferring resistance to diseases upon the wheat plant comprises a trichothecene 3-O-acetyltransferase gene.

Another embodiment of the present invention encompasses a wheat plant, or parts thereof, comprising the genotype of the wheat event JOPLIN1, wherein the genotype comprises the nucleotide sequence of SEQ ID NO: 1, SEQ ID NO: 2, SEQ ID NO: 3, SEQ ID NO: 4, SEQ ID NO: 7, SEQ ID NO: 8, or the complements thereof. In a further embodiment, the wheat plant is from wheat varieties COKER 9474, COKER 9803, COKER 9184, COKER 9152, COKER 9295, COKER 9312, COKER 9375, COKER 9436, Beretta, Savage, Natchez, Shelby, Crawford, Mason, Longhorn, Coronodo, Mallard and Patton, Dumas, Thunderbolt, Coronado, Ogallala, Longhorn, Cutter, Jagalene, TAM-111, Mason, and Natchez. One skilled in the art will recognize however, that the JOPLIN1 genotype can be introgressed into any plant variety that can be bred with wheat, including wild species, and thus the preferred varieties of this embodiment are not meant to be limiting.

One skilled in the art will further recognize that the transgenic genotype of the present invention can be introgressed by breeding into other wheat varieties comprising different transgenic genotypes. Such different transgenic genotypes provide for a trait such as, but not limited to, insect resistance, disease resistance, stress tolerance, herbicide tolerance or any combination thereof. Examples of such herbicide tolerant wheat plants include the glyphosate tolerant wheat event 33391 which is known in the art. It will be further recognized that other combinations can be made with the transgenic genotype of the invention and thus these examples should not be viewed as limiting.

One skilled in the art will also recognize that transgenic wheat seed comprising the transgenic genotype of the present invention can be treated with various seed-treatment chemicals, including fungicides, to augment or syngergize the disease resistance of the trichothecene 3-O-acetyltransferase. Such a combination may be used to increase the spectrum of activity and to increase the efficacy of the expressed protein and chemical.

The polynucleotides of the present invention can be used as molecular markers in a marker assisted breeding (MAB) method. Polynucleotides of the present invention can be used in methods, such as, AFLP markers, RFLP markers, RAPD markers, SNPs, and SSRs that identify genetically linked agronomically useful traits as described by Walton, Seed World 22 29 (July, 1993), the entirety of which is herein incorporated by reference; Burow and Blake, Molecular Dissection of Complex Traits, 13 29, Eds. Paterson, CRC Press, New York (1988), the entirety of which is herein incorporated by reference. The improved disease tolerance trait of wheat plant JOPLIN1 can be tracked in the progeny of a cross with wheat plant JOPLIN1 and any other wheat cultivar or variety using the MAB methods. The DNA molecules are markers for this trait and in MAB methods that are well known in the art can be used to track disease tolerance in wheat where wheat plant JOPLIN1 was a parent or ancestor.

As is readily apparent to one skilled in the art, the foregoing are only some of the various ways by which a wheat plant of the present invention can be obtained by those looking to introgress the transgenic genotype of the wheat event JOPLIN1 into other wheat lines. Other means are available, and the above examples are illustrative only.

The invention will be further described by reference to the following detailed examples. These examples are provided for purposes of illustration only, and are not intended to be limiting unless otherwise specified. Standard recombinant DNA and molecular cloning techniques used here are well known in the art and are described by Ausubel (ed.), Current Protocols in Molecular Biology, John Wiley and Sons, Inc: (1994); J. Sambrook, et al., Molecular Cloning: A Laboratory Manual, 3d Ed., Cold Spring Harbor, N.Y.: Cold Spring Harbor Laboratory Press (2001); and by T. J. Silhavy, M. L. Berman, and L. W. Enquist, Experiments with Gene Fusions, Cold Spring Harbor Laboratory, Cold Spring Harbor, N.Y. (1984).

In the description and examples reference is made to the following sequences:

| | |
|---|---|
| SEQ ID NO: 1 | JOPLIN1 5′ Genome + Insert sequence. Nucleotides 1 to 1393 is wheat |
| | genomic sequence (i.e. 5′ flanking sequence). Nucleotides 1394 to 1788 is |
| | insert DNA sequence. |
| SEQ ID NO: 2 | JOPLIN1 3′ Insert + Genome sequence. Nucleotides 1 to 426 is insert DNA |
| | sequence. Nucleotides 427 to 2471 is wheat genomic sequence (i.e. 3′ flanking |
| | sequence). |
| SEQ ID NO: 3 | JOPLIN1 5′ Genome-Insert junction. The first 15 nucleotides is wheat genome |
| | DNA sequence and the second 15 nucleotides is insert DNA sequence. |
| SEQ ID NO: 4 | 3′ Insert-Genome junction. The first 15 nucleotides is insert DNA sequence |
| | and the second 15 nucleotides is wheat genome DNA sequence. |
| SEQ ID NO: 5 | Insert sequence primer. |
| SEQ ID NO: 6 | 3′ Genome sequence primer. |
| SEQ ID NO: 7 | 5′ Genome - Insert-3′ Genome sequence. |
| SEQ ID NO: 8 | 5′ Genome - Insert-3′ Genome sequence. |

### EXAMPLE 1

### Polynucleotide Synthesis

Various polynucleotides based on the sequence information as described in the Sequence Listing can be synthesized chemically using standard techniques well known to the person skilled in the art. These polynucleotides include the primer sequences depicted in the Sequence Listing.

### EXAMPLE 2

### Transformation of Wheat

Transformation of wheat using a particle bombardment technique was carried out using a construct comprising the expression cassette contained within the sequence depicted as SEQ ID NO: 7 (nucleotides 1565 to 5290). This cassette comprises maize ubiquitin promoter-maize ubiquitin intron-trichothecene 3-O-acetyltransferase from F. sporotrichioides-nos terminator. In addition, wheat plants were also bombarded with an expression cassette derived from plasmid pCIB9818, as described in WO 00/60061. This plasmid is a 6111 base pair circular plasmid and the cassette used comprises the maize ubiquitin promoter (including a portion of the exon and intron) operably linked to the phosphate mannose isomerase selectable marker, an inverted PEPC intron#9 and a termination sequence from the CaMV 35S gene.

Immature zygotic wheat embryos were isolated at approximately 11 to 14 days post-anthesis (dpa) from surface sterilized caryopses and are pre-incubated on 3MS3S medium (Murashige and Skoog salts, MS vitamin, 300 mg/l glutamine, 150 mg/l asparagine, 3 mg/l 2,4-D, 30 g/l sucrose, and 7 g/l phytagar, pH at 5.8) for about 5 to 7 days in the dark at 22° C. Those embryos displaying the required embryonic phenotype were transferred to plasmolysis medium (3MS as above with 15 g/l of maltose in place of the sucrose) for about 3 to 4 hours in the dark pre-bombardment. Bombardment was carried out twice using 650 psi rupture disks to accelerate particles coated with approximately 1 mg/2 shots of 0.3 μM gold (coated using 0.17-0.67 μg DNA/2 shots). The shock wave is dissipated by a stainless steel mesh baffle positioned above the target plate. Post-bombardment, embryos were incubated in plasmolysis medium approximately 24 hours. Embryos were then incubated in callus initiation medium (3MS3S) for about 4 weeks in the dark without subculturing after which the resultant calli were dissected into approximately 2 to 5 mm pieces. These pieces of calli were incubated on regeneration/selection medium NG1M.5S (10 g/l of mannose, 5 g/l of sucrose, 5 mg/l GA3 and 1 mg/l NAA) for about 2 to 3 days in the dark and then in the light for about 2 weeks. Whole callus pieces were transferred to shoot and root initiation/selection medium MS2S.5M (MS salts and MS vitamins with 5 g/l mannose and 20 g/l sucrose) for about 3 to 4 weeks and then onto root initiation/selection medium ½ MS0S1.5M+0.5NAA (Half strength of MS salts and MS vitamin, 15 g/l mannose and 0.5 mg/l NAA) for a further 3 to 4 weeks. Resultant plants were moved to a greenhouse when they were large enough and have adequate roots. Plants were transplanted to soil in the greenhouse and grown to maturity.

### 3.1 Sample Preparation & PCR

The tissue samples for PCR detection of specific events can be prepared by any of the routine plant DNA extraction protocols. For example, 100 mg of wheat leaf material are harvested from the plant to be analyzed and processed according to Qiagen's DNEasy Plant Miniprep Kit. In a typical PCR reaction, approximately 1 μg of DNA is used and the reactions are carried out using standard methods. The reaction mixture, in addition to the sample DNA, may include a final concentration of 1×Clontech Advantage 2™ Taq Polymerase Buffer, 0.25 mM dNTP mixture, 1 μM of each primer used and 1× Clontech Advantage 2™ Taq Polymerase enzyme.

The PCR conditions are set to, for example, a denaturing cycle of 94° C. for 3 minutes, then 35 amplification cycles of 94° C. for 15 seconds; 60° C. for 30 seconds; and 72° C. for 45 seconds, followed by a final extension of 72° C. for 5 minutes. The PCR product is visualized on a 1% agarose gel-by-gel electophoresis. A positive result is indicated by a PCR product of the correct size.

Further confirmation of the identity of the PCR product, is obtained by sequencing using probes used to amplify the product.

A further detection method involves the use the PCR product as a hybridization probe for Southern Blot detection. The genomic DNA from the wheat plant to be analyzed can be digested with a restriction endonuclease. By employing a standard Southern Blot protocol with this DNA, the PCR product will give a unique hybridization signal when probed against the specific event.

### 3.2 Mapping of Insertion Sites for JOPLIN1

The position of the inserted DNA is determined using TAIL PCR (thermal asymmetric interlaced PCR), which is able to recover the sequence flanking the insertion at both the left and right border of the inserted DNA. A protocol for carrying out the TAIL PCR reaction is detailed, inter alia, in Liu et al., 1995 (The Plant Journal, vol. 8(3), pages 457 to 463). Once determined, knowledge of the flanking sequence and insertion sites can be used to design primers for event specific identification. Such primers are useful in, for example, breeding techniques in which the Event 1 trait is transferred to a different background germplasm.

The 5′ Genomic+insert sequence is set forth in SEQ ID NO: 1. Nucleotides 1 to 1393 of SEQ ID NO: 1 is the genomic sequence and nucleotides 1394 to 1788 of SEQ ID NO: 1 is the insert sequence. The 3′ Insert+genomic sequence is set forth in SEQ ID NO: 2. Nucleotides 1 to 426 of SEQ ID NO: 2 is the insert sequence and nucleotides 427 to 2471 of SEQ ID NO: 2 is the genomic sequence. SEQ ID NO: 8 sets forth the 5′ wheat genomic sequence flanking the insert, the entire insert sequence comprising

### 3.3 Event Specific Identification by PCR

Samples of plant material were prepared using techniques well known in the art. PCR reactions well known in the art were used. PCR conditions are as described above. Identification of insertion of terminator (3″) end of the inserted expression cassette was determined using the primers set forth in SEQ ID NO: 5 and SEQ ID NO: 6. Results were visualized using gel electrophoresis in accordance with protocols well known to the person skilled in the art. The size of the resulting amplicon was approximately 877 bp.

### EXAMPLE 4

### Immunostrip Assays

This example describes the use of immunostrip assays to test for the presence of trichothecene 3-O-acetyltransferase in a sample.

### 4.1 Procedure for Preparing Extract

Two, approximately 1 cm², discs of leaf tissue were cut from a leaf of the plant to be tested. Approximately 0.4-0.5 ml of extraction buffer was added and the tissue extracted using a plastic stirrer until a green extract was formed and the tissue was fibrous.

### 4.2 Preparation of Immunostrip

Briefly, the lateral-flow immunostrip comprises a detection membrane of nitrocellulose (2.5×18 cm) supported on a plastic backing (Arista™ brand plastic cassettes, Bethlehem, Pa.), in which a 1 mm line of specific mouse anti-trichothecene 3-O-acetyltransferase monoclonal antibody is sprayed. A reagent control line of donkey anti-mouse antibody is sprayed in parallel above the first antibody line. The bottom end portion of the strip of nitrocellulose is overlayered with a piece of treated polyester strip. The polyester strip is first treated with a solution B (90.5% BSA, 0.5% polyvinylalcohol and 0.1% Triton X-10; 50 mM phosphate buffer pH 7.4) and the colloidal gold conjugated mouse anti trichothecene 3-O-acetyltransferase antibody. The polyester strip is allowed to dry and is then overlayered with a sample application pad of cotton, which has been pretreated with a solution C (0.1% Triton X-100 and 0.1M borate buffer pH 8.5) and allowed to dry. Flanking the other end, or top end, of the nitrocellulose strip is another cotton pad to absorb the solution from the sample after it passes over the test antibody and control antibody areas on the nitrocellulose. The completed immunostrip may then be cut into smaller test strips.

### 4.3 Assay for Trichothecene 3-O-Acetyltransferase Using Immunostrips

The assay is performed by placing the bottom of an immunostrip described in Example 4.2 into 400 μl of extracted tissue. After waiting approximately 5 to 10 minutes, the results appeared. If trichothecene 3-O-acetyltransferase is present in the sample, a double red line appears: the lower line indicates the presence of trichothecene 3-O-acetyltransferase while the upper line is the control line signaling a properly working device. If no trichothecene 3-O-acetyltransferase is present, only a single red control line appears.

### EXAMPLE 5

### Fungal Resistance Assays

JOPLIN1 in a background germplasm of 98S0055-03 (AgriPro) was tested against the same germplasm with no trichothecene 3-O-acetyltransferase gene and against two commercial wheat varieties, AC Barrie and Alsen.

Test and control plants were grown in the field at a number of sites throughout the US and Canada. At each site, 8 replicates each of JOPLIN1 and the three control lines were planted, each replicate in a standard plot size of 5′ by 12′ (approximately 1.5 m by 3.7 m). The seeding rate was approximately 50 g/plot. Four of the replicates were artificially inoculated with Fusarium graminearum spores, the other four were left for natural infestation.

For artificial inoculation, Fusarium graminearum cultures may be grown as described in WO 00/60061. The spores were applied to the plants in a spray with a concentration of 50,000 spores/ml. Approximately 10 ml of spore suspension was used per foot of row (approximately 0.3 m) and the spores were sprayed onto the wheat spikes in two directions using a 110° flat fan with the filter removed. Spraying was performed in the late afternoon/evening to maximize disease. The spores were applied at anthesis, when the majority of the plants had reached Zadoks scale GS 6.5 (plot is at 50% anthesis) and then again, three days after the first inoculation.

At harvest, 5-foot (approximately 1.5 m) sections were harvested from the two center rows of each plot, by hand, in order to obtain approximately 200 g of grain sample. Disease was evaluated by counting the number of Fusarium damaged kernels (FDK) in the sample and by assaying for mycotoxin contamination. FDK is a visual trait and is used by grain traders to grade wheat quality. For assaying mycotoxin contamination, a 50 g grain sample was ground to a fine powder in a mill and the concentration of DON determined using a commercially available test (e.g. DONtest TAG™ mycotoxin testing system; Trilogy Analytical Laboratory, Inc., Washington, Mo.).

The following table shows the results of the field trials and is the average data obtained from the 10 locations where the trial was carried out.

TABLE 1
Percent of infected grain and mycotoxin concentration.

| Genotype | % FDK | DON Concentration (ppm) |
|---|---|---|
| 98S0055-03 + Event 1 | 7.2 | 12.3 |
| 98S0055-03 − Event 1 | 9.6 | 17.7 |
| AC Barrie | 8.2 | 13.0 |
| Alsen | 7.0 | 14.6 |

The field trials were repeated and the DON concentration was again analyzed. The results are shown in Table 2. In this table, trials 1 to 5 are carried out using natural infestation and trials 6 to 14 using inoculation.

TABLE 2
DON concentration (ppm).

| Trial | 98S0055-03 + Event 1 | 98S0055-03 − Event 1 | AC Barrie | Alsen |
|---|---|---|---|---|
| 1 | 1.73 | 2.67 | 3.80 | 2.54 |
| 2 | 1.16 | 3.21 | 2.95 | 4.49 |
| 3 | 0.75 | 2.11 | 3.05 | 2.20 |
| 4 | 1.88 | 4.63 | 4.36 | 4.65 |
| 5 | 0.40 | 1.47 | 1.54 | 2.17 |
| 6 | 1.73 | 3.56 | 5.38 | 3.29 |
| 7 | 4.09 | 9.42 | 8.85 | 8.94 |
| 8 | 0.39 | 1.39 | 1.92 | 1.44 |
| 9 | 1.03 | 2.46 | 4.22 | 4.91 |
| 10 | 0.54 | 4.32 | 4.86 | 8.97 |
| 11 | 2.42 | 5.26 | 6.41 | 8.22 |
| 12 | 0.56 | 1.63 | 1.43 | 1.88 |
| 13 | 3.34 | 8.62 | 7.81 | 9.19 |
| 14 | 1.12 | 2.11 | 2.53 | 2.55 |
| Mean | 1.51 | 3.78 | 4.22 | 4.67 |

In both cases, it can be seen that the DON concentration is decreased in wheat containing Event 1 when compared to the same germplasm lacking the event and to the two commercial standards. In the first trial, it can be seen that the % FDK is reduced compared to the same wheat line without Event 1 and is the same as or lower than the % FDK in the two commercial standards Alsen and AC Barrie.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the present invention.

### Deposited Cell Lines

The following cell lines were deposited under the rules of the Budapest Treaty at the DSMZ-Deutsche Sammlung von Mikroorganismen und Zellkulturen GmbH, Mascheroder Weg 1b, D-38124 Braunschweig, Germany.

| Cell Line | Accession No. |
|---|---|
| TRI-R MAb70 | DSM ACC2679 |
| TRI-R MAb72 | DSM ACC2680 |

What is claimed is:

1. An isolated polynucleotide comprising at least 20 contiguous nucleotides from the wheat event JOPLIN1, wherein a first half of the contiguous nucleotides is heterologous DNA sequence inserted into the wheat plant genome of event JOPLIN1 and a second half of the contiguous nucleotides is wheat plant genome DNA sequence flanking the point of insertion of a heterologous DNA sequence inserted into the wheat plant genome of event JOPLIN1.

2. An isolated polynucleotide which comprises at least 18 contiguous nucleotides of a nucleotide sequence selected from the group consisting of SEQ ID NO: 3, SEQ ID NO: 4, and complements thereof.

3. The isolated polynucleotide according to claim 2 which comprises at least 35 contiguous nucleotides of a nucleotide sequence selected from the group consisting of nucleotides 1364 to 1423 of SEQ ID NO: 1, nucleotides 397 to 456 of SEQ ID NO: 2, and the complements thereof.

4. The isolated polynucleotide according to claim 2 which comprises:
a) at least 50 nucleotides of SEQ ID NO: 1 including nucleotides 1393 and 1394; or
b) at least 50 nucleotides of SEQ ID NO: 2 including nucleotides 426 and 427.

5. The isolated polynucleotide according to claim 1 comprising a nucleotide sequence selected from the group consisting of SEQ ID NO: 1, SEQ ID NO: 2, SEQ ID NO: 3, SEQ ID NO: 4, and complements thereof.

6. A transgenic wheat plant comprising a polynucleotide according to claim 1.

7. Transgenic seed of the transgenic wheat plant according to claim 6 which comprises the polynucleotide.

8. The transgenic wheat plant according to claim 6, wherein the plant comprises SEQ ID NO: 1, SEQ IS NO: 2, SEQ ID NO: 3, SEQ ID NO: 4, SEQ ID NO: 7 or SEQ ID NO: 8.

9. A polynucleotide primer sequence for detecting wheat event JOPLIN1 nucleic acid in a sample comprising:
a) at least 10 contiguous nucleotides from nucleotides 1 to 1393 of SEQ ID NO: 1, or the complement thereof; or
b) at least 10 contiguous nucleotides from nucleotides 427 to 2471 of SEQ ID NO: 2, or the complements thereof.

10. The primer according to claim 9 comprising SEQ ID NO: 6 or the complement thereof.

11. A pair of polynucleotide primers comprising a first polynucleotide primer and a second polynucleotide primer which function together in the presence of a wheat event JOPLIN1 nucleic acid template in a sample to produce an amplicon diagnostic for the wheat event JOPLIN1, wherein the first primer sequence is or is complementary to a wheat plant genome flanking the point of insertion of a heterologous DNA sequence inserted into the wheat plant genome of wheat event JOPLIN1, and the second polynucleotide primer sequence is or is complementary to the heterologous DNA sequence inserted into the wheat plant genome of the wheat event JOPLIN1.

12. The pair of polynucleotide primers according to claim 11, wherein the first polynucleotide primer comprises at least 10 contiguous nucleotides from nucleotides 1 to 1393 SEQ ID NO: 1, or the complements thereof.

13. The pair of polynucleotide primers according to claim 11, wherein the first polynucleotide primer comprises least 10 contiguous nucleotides from nucleotides 427 to 2471 of SEQ ID NO: 2, or the complements thereof.

14. The pair of polynucleotide primers according to claim 11, wherein the second polynucleotide primer comprises at least 10 contiguous nucleotides selected from the group of nucleotide sequences consisting of nucleotides 1394 to 1788 of SEQ ID NO: 1, nucleotides 1 to 426 of SEQ ID NO: 2, nucleotides 1393 to 5512 of SEQ ID NO: 7, and complements thereof.

15. The pair of polynucleotide primers according to claim 14, wherein the second polynucleotide primer comprises SEQ ID NO: 5 or the complement thereof.

16. A method of detecting the presence of wheat event JOPLIN1 nucleic acids in a biological sample, the method comprising:
a) contacting the sample with a probe that hybridizes under high stringency conditions with genomic DNA from wheat event JOPLIN1 and does not hybridize under high stringency conditions with DNA of a control wheat plant;
b) subjecting the sample and probe to high stringency hybridization conditions; and
c) detecting hybridization of the probe to the DNA.

17. The method according to claim 16, wherein the probe comprises SEQ ID NO: 1, SEQ ID NO: 2, SEQ ID NO: 3, SEQ ID NO: 4, SEQ ID NO: 7, SEQ ID NO: 8, or complements thereof.

18. A method of detecting the presence of nucleic acids from the wheat plant of claim 6 in a biological sample, comprising:
a) contacting the sample with a first polynucleotide primer and a second polynucleotide primer that function together in a nucleic acid amplification reaction in the presence of a nucleic acid template from wheat event JOPLIN1 to produce an amplicon diagnostic for the wheat event JOPLIN1;
b) performing a nucleic acid amplification reaction, thereby producing the amplicon; and
c) detecting the amplicon.

19. The method of claim 18 wherein the amplicon comprises a nucleotide sequence selected from the group consisting of SEQ ID NO: 1, SEQ ID NO: 2, SEQ ID NO: 3, SEQ ID NO: 4, SEQ ID NO: 7, SEQ ID NO: 8, and complements thereof.

20. A method for detecting the wheat plant according to claim 6 which contains the polynucleotide of SEQ ID NO: 1 comprising:
a) preparing a sample containing the genomic DNA of the plant to be tested;
b) designing a pair of primers which are suitable for use in an amplification reaction to amplify a sequence comprising at least 18 contiguous nucleotides of SEQ ID NO: 3 and the complement thereof;
c) contacting the sample with the pair of primers;
d) performing an amplification reaction; and
e) detecting the resulting amplified sequence.

21. A method for detecting the wheat plant according to claim 6 which contains the polynucleotide of SEQ ID NO: 2 comprising:
a) preparing a sample containing the genomic DNA of the plant to be tested;
b) designing a pair of primers which are suitable for use in an amplification reaction to amplify a sequence comprising at least 18 contiguous nucleotides of SEQ ID NO: 4 and the complement thereof;
c) contacting the sample with the pair of primers;
d) performing an amplification reaction; and
e) detecting the resulting amplified sequence.

22. A method for detecting the plant according to claim 6 which comprises the nucleotide sequence of SEQ ID NO: 1 or the nucleotide sequence of SEQ ID NO: 2, or both, said method comprising:
a) preparing a sample containing the genomic DNA of the plant to be tested;
b) contacting the sample with at least one probe which is capable of hybridizing under high stringency hybridization and wash conditions to a sequence selected from the group consisting of a sequence comprising at least 18 contiguous nucleotides of SEQ ID NO: 3 and a sequence comprising at least 18 contiguous nucleotides of SEQ ID NO: 4;
c) subjecting the sample and at least one probe of step (b) to high stringency hybridization and wash conditions; and
d) detecting the thus hybridized probe to identify if the sample contains the polynucleotide.

23. A method for detecting the wheat plant according to claim 6 which comprises a protein capable of being encoded by the nucleotide sequence of SEQ ID NO: 7 said method comprising:
a) preparing a protein-extract of the plant to be tested;
b) providing an antibody which is capable of binding to a trichothecene 3-O-acetyltransferase protein;
c) contacting the extract with the antibody under conditions which allow the antibody to bind to the protein within the extract; and
d) detecting bound antibody to identify if the extract contains said protein.

24. A kit for detecting the presence of JOPLIN1 nucleic acids in a biological sample, wherein the kit comprises:
a) at least one nucleic acid molecule of sufficient length of contiguous nucleotides that is or is complementary to a nucleotide sequence selected from the group consisting of SEQ ID NO: 1, SEQ ID NO: 2, SEQ ID NO: 3, SEQ ID NO: 4, and fragments thereof, that functions as a DNA primer or probe specific for wheat event JOPLIN1; and
b) other materials necessary to enable nucleic acid hybridization or amplification.

25. A dipstick for use in the method of claim 23 comprising an anti-trichothecene 3-O-acetyltransferase antibody.

26. The dipstick of claim 25 comprising:
a) a test line of specific anti-trichothecene 3-O-acetyltransferase antibody;
b) a reagent control line of anti-mouse antibody;
c) a pad containing dried colloidal gold labeled anti-trichothecene 3-O-acetyltransferase antibody; and
d) a sample application pad.

27. A dipstick according to claim 26, wherein the anti-trichothecene 3-O-acetyltransferase antibody and the dried colloidal gold labeled anti-trichothecene 3-O-acetyltransferase antibody are independently selected from the group consisting of an antibody secreted by cell line DSM ACC 2679 and an antibody secreted by cell line DSM ACC 2680.


SEQUENCE LISTING (8 sequences)

SEQ ID NO: 1 - DNA, 1788 bp, Artificial Sequence
  5′ flank + insert from JOPLIN1 wheat
tgtggagaagcatagaccaacataatgtcactagtatgcctctacttgactagctcattaatcaaagatgattatgtttcctaaccatagacatgtgttgtcatttgattaacgggatcacatcattaggagaatggtgtgattgacatgacccattccgttagcctagcacttgatcgtttagtatattgctattgctttcttcatgacttatacatgttcctgtaactatgggattatgcaactcccgtttaccggaggaacactttgagtgctaccaaacgtcacaacgtaactgggtgattataaaggagtactacaggtgtctccaaaggtacatgttgggttggcgtatttcgagattaggttttgtcactccgattgtcagagaggtatctctgggccctctcggtaatgcacatcactataagccttgcaagcaatgtagctaatgagttagttacagaatgatgcattacgtaacgactaaagagacttgccggtaatgagttaaactaggtattagataccgacgattgaatctcgggcaagtaacataccgatgacaaagggaacaacgtatgttgttatgcggtttgaccgataaagatcttcgtagaatatgtaggagccaatatgagcatccaggttccgctattggttattgatcgagaatagttctaggtcatgtctacatagttctcgaacccgtagggtccgcacgcttaacgttatgatgacagttttattatgagtttatatattttgatcctttcgaccttggttccgtaggagcaagtcagaaaggttgagaaacagaaccatctgatttgattcgttcccaatagccatgagatgatcatcttagggtgatccttttgtcaacggatgctcctattacactcgtagtctctgaaggatgagaacccactatgtagcatctacatcgataattcaagcattgtatacgtcattagtccgattctttgtaggaactacccgtaataacgaacttgcaaaatggatctgtttatcataaagagattcattgttcctgaccctgcttcaccttaattgttatttgaacaaaaagatcacaataaacttttggtaaaagttctatcttggtcggagtggggatagcatttctcttctgcatgtctatggagttttgcaaaacccaaacacctcagagatagatatagaggtaggaatttgtcgaacgaaccacactccttcgtagacgtcaggagtccattgatgaaaaggggctggggaaagcttaaacccaagtcctacagtgatggatataagcgcaattgaaattcctggggagttatacatttgtgtattgataagaccgttcacaatttcttgaagctcgatctcccccctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatctaaactttactctacgaataatataatctatagtactacaataatatcagtgttttagagaatcatataaatgaacagttagacatgg

SEQ ID NO: 2 - DNA, 2471 bp, Artificial Sequence
  3′ insert + flank from JOPLIN1 wheat
cggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggagctccagcttttgttccctttagtgagggttaattgcgcgcttggcgtaatcatggtcatagctgtttcctgtgtgaaattgttatccgctcacaattccactcaacatacgagccggaagcataaagtgtaaagcctggggtgcctaatgagtgagctaactcacattaattgcgttgcgctcactgcccgctttccagtcgggaaacctgtcgtgccgtttttttcgctttttttctcccatcccatcttggacgggcccagaagcttttctcatgtttgaatggaggtcttccgcaaatgaatttccatataggaattatataatgtgcctgcagatattgattgaaagcttgtttcaattgaacgaggatgtgaacgagaaggcaacaaaagggagcttggagcaagaggagtcatttagtagttcacgaaaaaaaggcatagtagagaaagtctgaggagcaggaaaatttggtcacgtagatcttctatttcgccggaattcgttgattgctccgtactcatttacaatggaaaaactcctgttcgttgtaagatcactgaaggtcataaatttggagagtttgcttttacacggagacgaagaccctatcgaacaaatagaggaaaggggaaaaagtaaagtctaagcgacatatggatcaataaggaaatccaatttcggtaatccgaatcccatataggtcttttcttctctctcgaccagccctaggttggggataatgaatttttcgaatcttgttgggagatgcttcgctccgagtccgactgggacggaatgggggacccaggcccttcaatgcctccctccggatccactaactcggaagagctattcaaagcttttctggctgaccgtgaggacgcctctgcatcccgggagcagaccgctcccccggaagctgtggtcagtcaagagcggcatccgaatcccgctccaatgcctggagtggcagcggaagcagaacagaaccctccttcttttgattgggagggacgacctctctctctttgagaggaaagttctctacaaaaggcccgcgcctgtcacgcccagattctggacacattgcgtgatattactctgaagaacggagaggagctccgtgaagaagactatcgcaccatgcttgatttcatgttgagccgactccagtccgacgtcaatccccgggcgctgaaggagctcctgaacagactccaaaacggtcgggcacgatctcaaacttctagggcggcaaggagattcctcgacgaggactagtgaaactttttttgagagagaaatttccagtcatatcaggaatcagtacaaagtagttgacccttacaagcacactgaaatgcaaacacaaaagatcatgaacacctagagtaccctaaaacaaccataacacaagaagatctccggagccagtgtcatcatccatgaatcttgagagaagacccctgctgcagcagaaggatctgcaaccagtcgcaaacaggtcatcatcttcgaccacagtacaattgccgccacgctgcttcctcctttcttgacaccagcgctgagagggcatggacatcaatccaacacacctgcaacagccgtcgccatctttggctttgagtaccgcgaaaaatgtcccttccgaaaggaagagaactcgagtcattcgaccggtccagcaccgcggccgggccatccgcccgggcaaagcaggatctcttcaaaacttagagcatcaacaactagacttgcaaatccggcccctcaaacgatcgtgggcacgtccgtgtgcagtgaccggacacgtctcaaattttaacaggtgcatccggacatctcatattagattctcgaaractaaaataaacctacactacgtcggtcacctagctactcgtcatcagagawgtcgacgatctctgtgcccagctctggcagcatgggcggcagctgcagctccggctcctccgacggctccggctgctctgctccggcctcttccgcctctatctccgcgtcgagttcggcgaagagcgcgtcggacgccgcctgctcctgacagaggaacgcccggttttcctccacataggcctcatcctggatggcctgctgctcagccatctcctctgacggggcgacgctaaactccgtctccgcatgctccatgttgaaggccaacagctgctgctctggcttctccacctccatcagagccatcttctcctcctcttcccttsgmttcttcact

SEQ ID NO: 3 - DNA, 30 bp, Artificial Sequence
  5′ junction sequence from JOPLIN1 wheat
agctcgatctcccccctggcgaaaggggga

SEQ ID NO: 4 - DNA, 30 bp, Artificial Sequence
  3′ junction sequence from JOPLIN1 wheat
gaaacctgtcgtgccgtttttttcgctttt

SEQ ID NO: 5 - DNA, 23 bp, Artificial Sequence
  Insert primer chemically synthesized
gacctcgctttgaaccttttgag

SEQ ID NO: 6 - DNA, 21 bp, Artificial Sequence
  Genomic primer chemically synthesized
cgagaaggcaacaaaagggag

SEQ ID NO: 7 - DNA, 7557 bp, Artificial Sequence
  Genomic-Insert-Genomic from JOPLIN1 wheat
tgtggagaagcatagaccaacataatgtcactagtatgcctctacttgactagctcattaatcaaagatgattatgtttcctaaccatagacatgtgttgtcatttgattaacgggatcacatcattaggagaatggtgtgattgacatgacccattccgttagcctagcacttgatcgtttagtatattgctattgctttcttcatgacttatacatgttcctgtaactatgggattatgcaactcccgtttaccggaggaacactttgagtgctaccaaacgtcacaacgtaactgggtgattataaaggagtactacaggtgtctccaaaggtacatgttgggttggcgtatttcgagattaggttttgtcactccgattgtcagagaggtatctctgggccctctcggtaatgcacatcactataagccttgcaagcaatgtagctaatgagttagttacagaatgatgcattacgtaacgactaaagagacttgccggtaatgagttaaactaggtattagataccgacgattgaatctcgggcaagtaacataccgatgacaaagggaacaacgtatgttgttatgcggtttgaccgataaagatcttcgtagaatatgtaggagccaatatgagcatccaggttccgctattggttattgatcgagaatagttctaggtcatgtctacatagttctcgaacccgtagggtccgcacgcttaacgttatgatgacagttttattatgagtttatatattttgatcctttcgaccttggttccgtaggagcaagtcagaaaggttgagaaacagaaccatctgatttgattcgttcccaatagccatgagatgatcatcttagggtgatccttttgtcaacggatgctcctattacactcgtagtctctgaaggatgagaacccactatgtagcatctacatcgataattcaagcattgtatacgtcattagtccgattctttgtaggaactacccgtaataacgaacttgcaaaatggatctgtttatcataaagagattcattgttcctgaccctgcttcaccttaattgttatttgaacaaaaagatcacaataaacttttggtaaaagttctatcttggtcggagtggggatagcatttctcttctgcatgtctatggagttttgcaaaacccaaacacctcagagatagatatagaggtaggaatttgtcgaacgaaccacactccttcgtagacgtcaggagtccattgatgaaaaggggctggggaaagcttaaacccaagtcctacagtgatggatataagcgcaattgaaattcctggggagttatacatttgtgtattgataagaccgttcacaatttcttgaagctcgatctcccccctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatctaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccagaattcgtgatcaaatggccgcaacaagcagcacaagcagccagtcttttgacatagagctcgacatcatcggccagcaaccgcctcttctttcaatctacacccagatcagtctcgtttaccccgtctctgatccctcccagtatcccaccatcgtcagcacccttgaggaaggcctaaaacgcctctctcaaaccttcccatgggtcgcgggccaggtcaagaccgagggcatcagcgaaggaaacacaggaacttccaagatcattccatatgaggagacaccccgtcttgtggtgaaagacctccgtgatgattcctcagcgccaacgatcgaggggttgagaaaggcgggtttccccttagagatgtttgacgagaacgtcgtcgctccgaggaagacattagctatcggacctggcaatggccccaacgacccgaagcctgtgttgctattgcagctcaacttcattaagggcggactcattctcaccgtcaacggacaacatggtgctatggacatgacaggacaagatgcaattattcgtcttctctccaaggcgtgccgcaacgaatcattcaccgaggaggaaatctcggccatgaacctcgatcgcaagacggtagtccctctccttgaaaactacaaagttggtcctgagctagaccaccagatcgccaaacctgcgcctgctggcgacgctccacccgcaccggccaaggcaagctgggcgttcttttcattcactcccaaggccctctcggagctgaaagacgcagccacaaagactcttgacgcgtcgtccaagtttgtgtcaactgatgatgctctttcggcgtttatctggcaatcaacctcgcgcgtacgtctcgcaagattggatgcttccacacctactgaattctgccgcgctgtcgacatgcggggcccaatgggcgtatcaagcacatacccaggccttcttcaaaacatgacctaccatgactcgaccgtcgccgaaatcgccaacgaaccacttggcgcaacagcatcacgcctgcgctcggaactcaacagtgatcgtttgcgcagacgaacacaagctttggcgacgtacatgcatggcctgcctgacaagtcgagcgtctccctgaccgccgatgcgaatccgtcaagcagcatcatgctgagttcctgggccaaggtgggatgctgggagtatgactttgggtttggactgggtaagcctgagagtgtgagaagacctcgctttgaaccttttgagagtttgatgtactttatgcccaagaagcctgatggggagtttacggcgtccatttctctgagggatgaggatatggagagactaaaggcggatgaggagtggacaaagtacgcaaagtatattgggtagatagtttactagactactgcaggatatcgtggatccactagttctagagcggccgcgaatttccgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggagctccagcttttgttccctttagtgagggttaattgcgcgcttggcgtaatcatggtcatagctgtttcctgtgtgaaattgttatccgctcacaattccactcaacatacgagccggaagcataaagtgtaaagcctggggtgcctaatgagtgagctaactcacattaattgcgttgcgctcactgcccgctttccagtcgggaaacctgtcgtgccgtttttttcgctttttttctcccatcccatcttggacgggcccagaagcttttctcatgtttgaatggaggtcttccgcaaatgaatttccatataggaattatataatgtgcctgcagatattgattgaaagcttgtttcaattgaacgaggatgtgaacgagaaggcaacaaaagggagcttggagcaagaggagtcatttagtagttcacgaaaaaaaggcatagtagagaaagtctgaggagcaggaaaatttggtcacgtagatcttctatttcgccggaattcgttgattgctccgtactcatttacaatggaaaaactcctgttcgttgtaagatcactgaaggtcataaatttggagagtttgcttttacacggagacgaagaccctatcgaacaaatagaggaaaggggaaaaagtaaagtctaagcgacatatggatcaataaggaaatccaatttcggtaatccgaatcccatataggtcttttcttctctctcgaccagccctaggttggggataatgaatttttcgaatcttgttgggagatgcttcgctccgagtccgactgggacggaatgggggacccaggcccttcaatgcctccctccggatccactaactcggaagagctattcaaagcttttctggctgaccgtgaggacgcctctgcatcccgggagcagaccgctcccccggaagctgtggtcagtcaagagcggcatccgaatcccgctccaatgcctggagtggcagcggaagcagaacagaaccctccttcttttgattgggagggacgacctctctctctttgagaggaaagttctctacaaaaggcccgcgcctgtcacgcccagattctggacacattgcgtgatattactctgaagaacggagaggagctccgtgaagaagactatcgcaccatgcttgatttcatgttgagccgactccagtccgacgtcaatccccgggcgctgaaggagctcctgaacagactccaaaacggtcgggcacgatctcaaacttctagggcggcaaggagattcctcgacgaggactagtgaaactttttttgagagagaaatttccagtcatatcaggaatcagtacaaagtagttgacccttacaagcacactgaaatgcaaacacaaaagatcatgaacacctagagtaccctaaaacaaccataacacaagaagatctccggagccagtgtcatcatccatgaatcttgagagaagacccctgctgcagcagaaggatctgcaaccagtcgcaaacaggtcatcatcttcgaccacagtacaattgccgccacgctgcttcctcctttcttgacaccagcgctgagagggcatggacatcaatccaacacacctgcaacagccgtcgccatctttggctttgagtaccgcgaaaaatgtcccttccgaaaggaagagaactcgagtcattcgaccggtccagcaccgcggccgggccatccgcccgggcaaagcaggatctcttcaaaacttagagcatcaacaactagacttgcaaatccggcccctcaaacgatcgtgggcacgtccgtgtgcagtgaccggacacgtctcaaattttaacaggtgcatccggacatctcatattagattctcgaaractaaaataaacctacactacgtcggtcacctagctactcgtcatcagagawgtcgacgatctctgtgcccagctctggcagcatgggcggcagctgcagctccggctcctccgacggctccggctgctctgctccggcctcttccgcctctatctccgcgtcgagttcggcgaagagcgcgtcggacgccgcctgctcctgacagaggaacgcccggttttcctccacataggcctcatcctggatggcctgctgctcagccatctcctctgacggggcgacgctaaactccgtctccgcatgctccatgttgaaggccaacagctgctgctctggcttctccacctccatcagagccatcttctcctcctcttcccttsgmttcttcact

SEQ ID NO: 8 - DNA, 6023 bp, Artificial Sequence
  Genomic-Insert-Genomic from JOPLIN1 wheat
tgtggagaagcatagaccaacataatgtcactagtatgcctctacttgactagctcattaatcaaagatgattatgtttcctaaccatagacatgtgttgtcatttgattaacgggatcacatcattaggagaatggtgtgattgacatgacccattccgttagcctagcacttgatcgtttagtatattgctattgctttcttcatgacttatacatgttcctgtaactatgggattatgcaactcccgtttaccggaggaacactttgagtgctaccaaacgtcacaacgtaactgggtgattataaaggagtactacaggtgtctccaaaggtacatgttgggttggcgtatttcgagattaggttttgtcactccgattgtcagagaggtatctctgggccctctcggtaatgcacatcactataagccttgcaagcaatgtagctaatgagttagttacagaatgatgcattacgtaacgactaaagagacttgccggtaatgagttaaactaggtattagataccgacgattgaatctcgggcaagtaacataccgatgacaaagggaacaacgtatgttgttatgcggtttgaccgataaagatcttcgtagaatatgtaggagccaatatgagcatccaggttccgctattggttattgatcgagaatagttctaggtcatgtctacatagttctcgaacccgtagggtccgcacgcttaacgttatgatgacagttttattatgagtttatatattttgatcctttcgaccttggttccgtaggagcaagtcagaaaggttgagaaacagaaccatctgatttgattcgttcccaatagccatgagatgatcatcttagggtgatccttttgtcaacggatgctcctattacactcgtagtctctgaaggatgagaacccactatgtagcatctacatcgataattcaagcattgtatacgtcattagtccgattctttgtaggaactacccgtaataacgaacttgcaaaatggatctgtttatcataaagagattcattgttcctgaccctgcttcaccttaattgttatttgaacaaaaagatcacaataaacttttggtaaaagttctatcttggtcggagtggggatagcatttctcttctgcatgtctatggagttttgcaaaacccaaacacctcagagatagatatagaggtaggaatttgtcgaacgaaccacactccttcgtagacgtcaggagtccattgatgaaaaggggctggggaaagcttaaacccaagtcctacagtgatggatataagcgcaattgaaattcctggggagttatacatttgtgtattgataagaccgttcacaatttcttgaagctcgatctcccccctggcgaaagggggatgtgctgcaaggcgattaagttgggtaacgccagggttttcccagtcacgacgttgtaaaacgacggccagtgarykcgcgtaatacgactcactatagggcgaattgggtaccgggccccccctcgaggtcgacggtatcgataagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatytaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccagaattcgtgatcaaatggccgcaacaagcagcacaagcagccagtcttttgacatagagctcgacatcatcggccagcaaccgcctcttctttcaatctacacccagatcagtctcgtttaccccgtctctgatccctcccagtatcccaccatcgtcagcacccttgaggaaggcctaaaacgcctctctcaaaccttcccatgggtcgcgggccaggtcaagaccgagggcatcagcgaaggaaacacaggaacttccaagatcattccatatgaggagacaccccgtcttgtggtgaaagacctccgtgatgattcctcagcgccaacgatcgaggggttgagaaaggcgggtttccccttagagatgtttgacgagaacgtcgtcgctccgaggaagacattagctatcggacctggcaatggccccaacgacccgaagcctgtgttgctattgcagctcaacttcattaagggcggactcattctcaccgtcaacggacaacatggtgctatggacatgacaggacaagatgcaattattcgtcttctctccaaggcgtgccgcaacgaatcattcaccgaggaggaaatctcggccatgaacctcgatcgcaagacggtagtccctctccttgaaaactacaaagttggtcctgagctagaccaccagatcgccaaacctgcgcctgctggcgacgctccacccgcaccggccaaggcaagctgggcgttcttttcattcactcccaaggccctctcggagctgaaagacgcagccacaaagactcttgacgcgtcgtccaagtttgtgtcaactgatgatgctctttcggcgtttatctggcaatcaacctcgcgcgtacgtctcgcaagattggatgcttccacacctactgaattctgccgcgctgtcgacatgcggggcccaatgggcgtatcaagcacatacccaggccttcttcaaaacatgacctaccatgactcgaccgtcgccgaaatcgccaacgaaccacttggcgcaacagcatcacgcctgcgctcggaactcaacagtgatcgtttgcgcagacgaacacaagctttggcgacgtacatgcatggcctgcctgacaagtcgagcgtctccctgaccgccgatgcgaatccgtcaagcagcatcatgctgagttcctgggccaaggtgggatgctgggagtatgactttgggtttggactgggtaagcctgagagtgtgagaagacctcgctttgaaccttttgagagtttgatgtactttatgcccaagaagcctgatggggagtttacggcgtccatttctctgagggatgaggatatggagagactaaaggcggatgaggagtggacaaagtacgcaaagtatattgggtagatagtttactagactactgcaggatatcgtggatccactagttctagagcggccgcgaatttccgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagctgcgcraacwaggataaattatcgcgcgcggtgtcatctatgttactagatcgggagctccagcttttgttccctttagtgagggttaattscgmgcttggcagtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaracctgtcgtgccgtttttttcgctttttttctcccatcccatcttggacgggcccagaagcttttctcatgtttgaatggaggtcttccgcaaatgaatttccatataggaattatataatgtgcctgcagatattgattgaaagcttgtttcaattgaacgaggatgtgaacgagaaggcaacaaaagggagcttggagcaagaggagtcatttagtagttcacgaaaaaaggcatagtagagaaagtctgaggagcaggaaaatttggtcacgtagatcttctatttcgccggaattcgttgattgctccgtactcatttacaatggaaaaactcctgttcgttgtaagatcactgaaggtcataaatttggagagtttgcttttacacgggagacgaagaccctatcgaacaaatagaggaaaggggaaaaagtaaagtctaagcgacatatggatcaataaggaaatccaatttcggtaatccgaatccccatataggtcttttcttcwywctcgacc